SEQUENCE LISTING (30 sequences)

SEQ ID NO: 1 - DNA, 18 bp, Artificial Sequence
  Description of Artificial Sequence Synthetic oligonucleotide
ctcattcaataccctacg

SEQ ID NO: 2 - DNA, 18 bp, Artificial Sequence
  Description of Artificial Sequence Synthetic oligonucleotide
ttcaataccctacgtctc

SEQ ID NO: 3 - DNA, 22 bp, Artificial Sequence
  Description of Artificial Sequence Synthetic oligonucleotide
gagacgtagggtattgaatgag

SEQ ID NO: 4 - DNA, 17 bp, Artificial Sequence
  Description of Artificial Sequence Synthetic oligonucleotide
tcattcaataccctacg

SEQ ID NO: 5 - DNA, 18 bp, Artificial Sequence
  Description of Artificial Sequence Synthetic oligonucleotide
ttcaataccctacgtctc

SEQ ID NO: 6 - DNA, 26 bp, Artificial Sequence
  Description of Artificial Sequence Synthetic oligonucleotide
tggagacgtagggtattgaatgaggg

SEQ ID NO: 7 - DNA, 18 bp, Artificial Sequence
  Description of Artificial Sequence Synthetic oligonucleotide
ctcattcaataccctacg

SEQ ID NO: 8 - DNA, 18 bp, Artificial Sequence
  Description of Artificial Sequence Synthetic oligonucleotide
ttcaataccctacgtctc

SEQ ID NO: 9 - DNA, 18 bp, Artificial Sequence
  Description of Artificial Sequence Synthetic oligonucleotide
ttcaataccctacgtctc

SEQ ID NO: 10 - DNA, 18 bp, Artificial Sequence
  Description of Artificial Sequence Synthetic oligonucleotide
ctcattcaataccctacg

SEQ ID NO: 11 - DNA, 41 bp, Artificial Sequence
  Description of Artificial Sequence Synthetic oligonucleotide
ttcaataccctacgacctgggggagtattgcggaggaaggt

SEQ ID NO: 12 - DNA, 28 bp, Artificial Sequence
  Description of Artificial Sequence Synthetic oligonucleotide
ccccaggtcgtagggtattgaatgaggg

SEQ ID NO: 13 - DNA, 21 bp, Artificial Sequence
  Description of Artificial Sequence Synthetic oligonucleotide
tgggtcattcaataccctacg

SEQ ID NO: 14 - DNA, 18 bp, Artificial Sequence
  Description of Artificial Sequence Synthetic oligonucleotide
ttcaataccctacgtctc

SEQ ID NO: 15 - DNA, 35 bp, Artificial Sequence
  Description of Artificial Sequence Synthetic oligonucleotide
tggagacgtagggtattgaatgtgggcgggtgggt

SEQ ID NO: 16 - DNA, 18 bp, Artificial Sequence
  Description of Artificial Sequence Synthetic oligonucleotide
ttcaataccctacgtctc

SEQ ID NO: 17 - DNA, 66 bp, Artificial Sequence
  Description of Artificial Sequence Synthetic oligonucleotide
tgggtcaataccctacgataccctacgtctcttttttgagacgtagggtattgtgggcgggtgggt

SEQ ID NO: 18 - DNA, 20 bp, Artificial Sequence
  Description of Artificial Sequence Synthetic oligonucleotide
ccctcattcaataccctacg

SEQ ID NO: 19 - DNA, 20 bp, Artificial Sequence
  Description of Artificial Sequence Synthetic oligonucleotide
ttcaataccctacgtctcca

SEQ ID NO: 20 - DNA, 14 bp, Artificial Sequence
  Description of Artificial Sequence Synthetic oligonucleotide
ttcaataccctacg

SEQ ID NO: 21 - DNA, 16 bp, Artificial Sequence
  Description of Artificial Sequence Synthetic oligonucleotide
cattcaataccctacg

SEQ ID NO: 22 - DNA, 22 bp, Artificial Sequence
  Description of Artificial Sequence Synthetic oligonucleotide
tggagacgtagggtattgaatg

SEQ ID NO: 23 - DNA, 22 bp, Artificial Sequence
  Description of Artificial Sequence Synthetic oligonucleotide
ttcaataccctacgtctttttt

SEQ ID NO: 24 - DNA, 28 bp, Artificial Sequence
  Description of Artificial Sequence Synthetic oligonucleotide
ttttttgacgtagggtattgaatgaggg

SEQ ID NO: 25 - DNA, 10 bp, Artificial Sequence
  Description of Artificial Sequence Synthetic oligonucleotide
ctggacgtcg

SEQ ID NO: 26 - DNA, 10 bp, Artificial Sequence
  Description of Artificial Sequence Synthetic oligonucleotide
acgtcgcctg

SEQ ID NO: 27 - DNA, 40 bp, Artificial Sequence
  Description of Artificial Sequence Synthetic oligonucleotide
gtgcaggcgaccgaatgccatggcattcggcgtccagctc

SEQ ID NO: 28 - DNA, 60 bp, Artificial Sequence
  Description of Artificial Sequence Synthetic oligonucleotide
gtgcaggcgatgagcacgagctcgtgctcaccgaatgccatggcattcggcgtccagctc

SEQ ID NO: 29 - DNA, 18 bp, Artificial Sequence
  Description of Artificial Sequence Synthetic oligonucleotide
ttcaataccctacgtctc

SEQ ID NO: 30 - DNA, 18 bp, Artificial Sequence
  Description of Artificial Sequence Synthetic oligonucleotide
ttcaataccctacgtctc

(19) United States
(12) Patent Application Publication
(10) Pub. No.: US 2016/0033495 A1
(43) Pub. Date: Feb. 4, 2016

(54) Detection of Non-Nucleic Acid Analytes Using Strand Displacement Exchange Reactions

(71) Applicants: Kurt Vesterager GOTHELF, Risskov (DK); Zhai ZHANG, West Haven, CT (US); Unisense Diagnostics ApS, Aarhus N (DK)

(72) Inventors: Kurt Vesterager Gothelf, Risskov (DK); Zhao Zhang, West Haven, CT (US); Michael Brondum Kjelstrup, Aarhus (DK); Christian Hejesen, Aarhus (DK)

(73) Assignee: Unisense Diagnostics ApS, Aarhus N (DK)

(21) Appl. No.: 14/426,773

(22) PCT Filed: Sep. 11, 2013
(86) PCT No.: PCT/EP2013/068824

§ 371 Date: Mar. 9, 2015

(30) Foreign Application Priority Data
Sep. 11, 2012 (DK) PA 2012 70555

(51) Int. Cl. G01N 33/543; C12Q 1/68

(52) U.S. Cl. CPC G01N 33/54386; C12Q 1/6832

(57) ABSTRACT

The present invention relates to an analyte detection system for detecting analytes different from DNA and RNA. The system comprises a set of oligonucleotides which may hybridize to each other in specific ways and is able to generate a signal based on the specific hybridization events. The system relies on changes in the hybridization equilibrium between the oligonucleotides in the presence of an analyte or analytes, which results in a change in signal.

### TECHNICAL FIELD OF THE INVENTION

The present invention relates to an analyte detection system. In particular the present invention relates to an analyte detection system wherein the analyte is not nucleic acid, i.e. DNA and RNA.

### BACKGROUND OF THE INVENTION

A variety of different methods are available for the detection of peptides/proteins and other molecules in a sample, such as ELISA, SPR, QCM, electrochemical sensors etc. These surface-based methods are endowed with high sensitivity and multiplex sensing ability, but the immobilization procedure may interfere with protein-ligand binding and frequently requires cautious washing and blocking to combat nonspecific adsorption. There are only a handful of well-established homogeneous assays, among which fluorescence polarization is widely used to study small molecule-protein interactions (Analysis of protein-ligand interactions by fluorescence polarization, Ana Rossi & Colin Taylor, Nature protocols, 2011).

Electrochemical biosensors are normally based on enzymatic catalysis of a reaction that produces or consumes electrons. Enzyme-linked immunosorbent assays (ELISAs) are plate-based assays designed for detecting and quantifying substances such as peptides, proteins, antibodies and hormones. Surface plasmon resonance (SPR) detects the increase in size of an immobilized ligand when it binds a larger protein by recording the change in refractive index at the surface of a thin metal film. The quartz crystal microbalance (QCM) detection scheme is based on the measurement of the mass changes and physical properties of thin layers deposited on the crystal surfaces. Fluorescence polarization detects binding of a small fluorescent ligand to a larger protein using plan-polarized light to detect the change in effective molecular volume.

A range of assays also exist which can detect nucleic acid molecules in a sample, such as PCR, Southern blotting, Northern blotting, and FISH. A more recent approach is the DNA toehold exchange reaction. The DNA toehold exchange reaction is a system where two DNA strands each having a specific toehold region compete with each other to hybridize with a third DNA strand serving as a substrate. Owing to its great modularity, designability and sensitivity, it has been used to build catalytic DNA network (Zhang et al. Engineering entropy-driven reactions and networks catalyzed by DNA. Science 2007, 318, 1121-1125), control DNA strand displacement kinetics (Zhang et al. Control of DNA strand displacement kinetics using toehold exchange. J Am Chem Soc 2009, 131, 17303-17314), and optimize the specificity of nucleic acid hybridization (Zhang et al. Optimizing the specificity of nucleic acid hybridization. Nat Chem 2012, 4, 208-214). However, these systems are only relevant for detecting nucleic acid molecules.

Hence, an improved detection system for detecting molecules different from DNA and RNA would be advantageous, and in particular a more efficient and/or reliable detection system for detecting small molecules different from DNA and RNA would be advantageous.

### SUMMARY OF THE INVENTION

The present invention provides an analyte detection system for detecting analytes different from DNA and RNA. The system comprises a set of oligonucleotides which may hybridize to each other in specific ways and is able to generate a signal based on the specific hybridization events. The system relies on changes in the hybridization equilibrium between the oligonucleotides in the presence of an analyte or analytes, which results in a change in signal.

Herein we present a detection system exploiting the DNA toehold exchange reaction for non-nucleic acid target detection, by attaching a small molecule (e.g. antigen or hapten) onto one or more of the three DNA strands. The specific binding between the small molecule and its receptor protein or antibody shifts the original equilibrium, and the population change of each component in solution can be monitored by e.g. FRET (Förster resonance energy transfer) signal. The scheme is shown below:

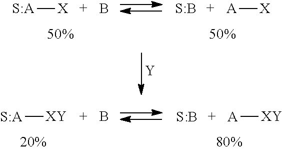

Here, A, B, and S are the three DNA oligonucleotides, among which both A and B are partially complementary to S. X represents the small molecule labelled on A, and Y is its corresponding binding protein. For example, without Y, A and B has equal or almost equal ability to hybridize with S, and with Y, the bulkiness, charge or other effects of the protein will affect the hybridization energy, thus their percentage will for example change from 50:50 to 20:80. Basically, this concept takes advantage of the fact that small perturbation of the energy for each hybrid formation will result in relatively large shifts in the equilibrium between the two DNA hybrids.

In a typical toehold exchange reaction system, two DNA strands sharing the same branch migration region but differing in the toehold region, will dynamically compete with each other to hybridize with a third DNA. A whole displacement cycle is illustrated in FIG. 2. For example, strand A (A) pairs with strand S (S) to form AS duplex at the beginning. Since S has another toehold region for strand B (B), the toehold on B has a chance to hybridize with the toehold on S. As soon as both A and B bind with S, branch migration process will occur, due to sequence symmetry. In this process, A and B have an equal chance to displace each other, so half of the products would be that B completely possess the branch migration region of S, while A only binds with S through the toehold. Since the toehold is normally short, the hybridization is unstable and transient, ultimately leaving the components of BS plus A in solution. All the steps above are reversible. Notice that here B typically has a labelled binding moiety in the branch migration region.

In the presence of an analyte, the binding of the analyte to the small molecule linked to B might not have big influence on toehold binding, but will cause an increased energetic barrier for B to perform the branch migration process, thus giving B a disadvantage (or occasionally advantage) compared to A in the competitive binding to S. Therefore, in the final products the thermodynamic equilibrium is shifted toward the direction of formation of more AS duplex (occasionally more BS duplex) as well as single-stranded B bound with target analyte, and this population change can be detected by FRET or other optical methods.

In the example section different types of studies show that the concept is indeed functional for detection analytes different from RNA and DNA.

In conclusion, a novel detection system has been developed which may detect both proteins and small molecules based on the fine-tuned toehold exchange reaction. This assay is enzyme-free, and compared to traditional assay such as ELISA, it circumvents protein modification and the effort of searching bivalent antigen/antibody.

Such assay may find use as a sensitive, specific, robust, and high-throughput platform for detection of various targets in health, food, veterinary and environmental related activities.

Thus, an object of the present invention relates to providing a detection system which may detect molecules different from DNA and RNA. Another object is to provide a detection system which may detect small molecules different from DNA and RNA and only requiring one binding site on the analyte to detect the analyte. This is in contrast to e.g. ELISA assays which require two binding sites on the analyte. For small analytes two binding sites may not be present.

Thus, one aspect of the invention relates to an analyte detection system for detecting analytes different from DNA and RNA, the system comprising at least a first oligonucleotide A, a second oligonucleotide B, and a third oligonucleotide S, wherein:
each of oligonucleotides A and B comprise a sequence that is complementary or partly complementary to a sequence on oligonucleotide S, and wherein oligonucleotides A and B compete for hybridization to oligonucleotide S in a dynamic equilibrium, and optionally wherein at least one of oligonucleotides A and B comprises a covalently linked binding moiety capable of interacting with an analyte different from DNA and RNA; and
at least one of oligonucleotides A and B, or a covalently linked binding moiety bound to said oligonucleotide, is capable of interacting with an analyte different from DNA and RNA, such that interaction of said analyte with the oligonucleotide or binding moiety results in a shift in the hybridization equilibrium, the shift in equilibrium providing a detectable signal.

Illustrative examples of the analyte detection system are presented in FIGS. 1 and 2.

As described in the background section an assay similar to the present one has been described for DNA detection. However, that setup is not suitable for detection of analytes different from DNA and RNA, such as proteins and small organic molecules. For such purposes different system are more suitable such as ELISA.

Another aspect of the present invention relates to a kit of parts comprising an analyte detection system according to the invention.

Yet another aspect of the present invention is to provide a kit of parts comprising the first oligonucleotide, the second oligonucleotide and the third oligonucleotide according to the present invention.

Still another aspect of the present invention is to provide a method for detection the presence or level of an analyte different from DNA and RNA in a sample, the method comprising
a) providing a sample comprising or suspected of comprising an analyte of interest;
b) providing the analyte detection system according to the present invention;
c) incubating the sample with the analyte detection system;
d) comparing the detected level of analyte to a reference level; and
e) determining the presence or level of analyte in the sample.

### BRIEF DESCRIPTION OF THE FIGURES

FIG. 1 shows a specific embodiment of the invention with specific sequences of the first oligonucleotide 1 (SEQ ID NO: 1), the second oligonucleotide 3 (SEQ ID NO: 2) and the third oligonucleotide 5 (SEQ ID NO: 3) according to the invention. 8, 8′ and 9, 9′ indicate the toehold regions and 7, 7′ indicate the branch migration region. Arrows indicate the 5′ to 3′ direction of the oligonucleotides.

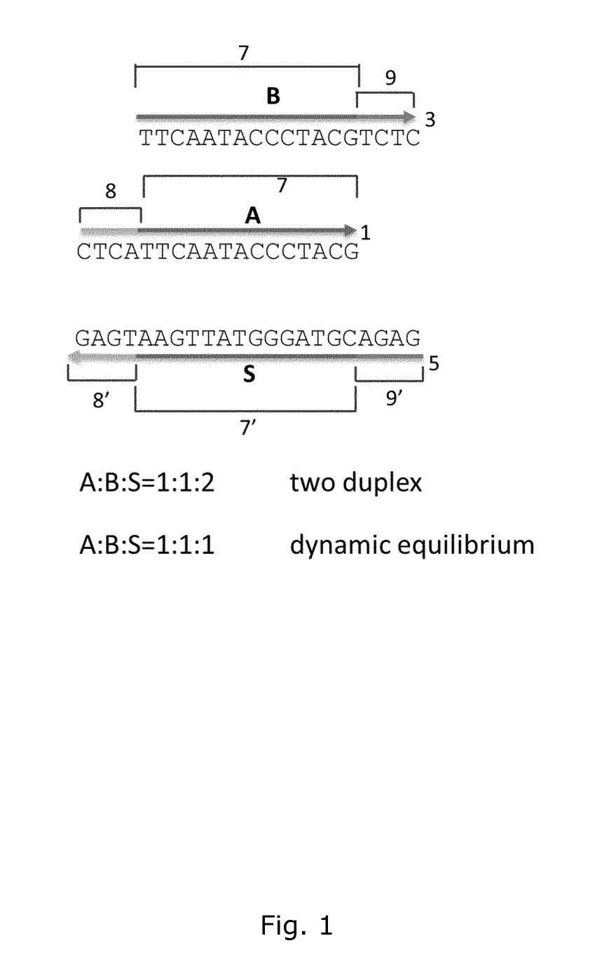

FIG. 2 shows an example of how the equilibrium changes when an analyte binds to the binding moiety (4). Numbering as indicated in FIG. 1. a) Without target analyte, A and B can be designed to have equal or close to equal probability to hybridize with S, resulting a 50/50 ratio for AS and BS. b) In the presence of analyte, the steric or electrostatic effect of the analyte (in this case the target) causes an energetic barrier for B to perform in the branch migration process, resulting a 80/20 ratio for AS and BS.

FIG. 3 shows the detection system when biotin is the binding moiety and streptavidin (STV) is the analyte. Specific sequences comprising modification of the first oligonucleotide 1 (SEQ ID NO: 4), the second oligonucleotide 3 (SEQ ID NO:5) and the third oligonucleotide 5 (SEQ ID NO: 6) according to the invention are presented. Numbering as indicated in FIG. 1. In addition 2 and 6 indicate the signalling system, and 4 indicates the binding moiety.

FIG. 4 shows the results of the three-strand system for streptavidin (STV) detection and for biotin detection. (a) Normalized FRET efficiency for two samples with and without STV respectively. (b) Fluorescence spectrum for two samples with and without STV respectively. (c) Titration curve of STV detection. (d) Non-denaturing gel analysis for STV detection assay. The first two lanes are systems with one biotin, while the last two lanes are systems with two biotins. The inset is the scheme of STV bound on strand B by two-sites interaction. (e) Quantitative detection of biotin by an inhibitive strategy. The analyte (biotin) is mixed with STV first, then the mixture is added to the assay. Analyte will take up the binding sites on STV, which is therefore inactivated.

FIG. 5 shows three strategies for the sensor assays. In Strategy 1 direct detection of the analyt/target (e.g. protein or antibody) is obtained by interaction with a binding moiety at the ABS system. In the inhibitive strategy 2, the unknown specimen and the corresponding protein are premixed, followed by addition to the normal assay. If the specimen includes free ligands, they will block the binding-sites of the protein, making it incapable of functioning on the assay. In the competitive strategy 3, the corresponding protein is first mixed with the normal DNA assay to form a new assay, then the unknown specimen is added to compete with the ligands on strand B to bind with proteins, thus having the opposite effect on the equilibrium.

FIG. 6 shows a control experiment. A) Influence of additives on the ABS system described in FIG. 3 but without binding moiety. Three fluorophore setup. B) Influence of additives on the ABS system described in FIG. 3 but with the biotin binding moiety. Two fluorophore setup. No detectable change is observed after adding any of the analytes except when adding streptavidin to the system containing the biotin binding moiety.

FIG. 7 shows the kinetics of the streptavidin binding assay. Comparison of two assays with toehold length of 4 nt (left) and 6 nt (right) respectively. In both assays, biotin modification locates on the toehold region of B, and A and S has a pair of fluorophores (Alexa488 & Alexa555). The results indicate that longer toehold (on both A and B) ensures faster equilibrium, at the expense of leading to a smaller effect of STV binding, which is confirmed by FRET results in (b). The final design is preferably a compromise of kinetics and signal-noise ratio. In addition, FRET measurement suggest 3 hours incubation is enough for the 4 nt system to reach equilibrium after adding STV (data not shown).

FIG. 8 illustrates the detection system when digoxigenin (DIG) is the binding moiety and anti-digoxigenin (aD) is the analyte. Figure A) shows specific sequences comprising modification of a first oligonucleotide 1 (SEQ ID NO: 7), a second oligonucleotide 3 (SEQ ID NO: 8) and a third oligonucleotide 5 (SEQ ID NO: 6) according to the invention. Numbering as indicated in FIG. 1. In addition 2 and 6 indicate the signalling system, and 4 indicates the binding moiety. Bar plot B) shows the raw FRET signal change upon detection of aD. C) chemical structure of digoxigenin. Graph D) shows the FRET signal change, upon titration of aD to oligonucleotides 1, 3 and 5. E) shows a fluorescence image of a native polyacrylamide gel, where only oligonucleotide A is visible. An increase in the AS population is seen upon addition of aD.

FIG. 9 illustrates the detection system in human plasma when digoxigenin (DIG) is the binding moiety and anti-digoxigenin (aD) is the analyte. Figure A) numbering is the same as FIG. 8A. Graph B) is the result of a triple controlled experiment comparing the setup of the sensor (ABS) without any added human plasma with samples with human plasma. Columns 1 and 3 show the FRET change upon detection of aD without human plasma, with column 2 and 5 showing the FRET change in human plasma. Columns 4 and 6 show competitive assays for the detection of free digoxigenin without and with human plasma respectively.

FIG. 10 illustrates the detection system in human saliva when digoxigenin (DIG) is the binding moiety and anti-digoxigenin (aD) is the analyte. Figure A) numbering is the same as FIG. 8A. Bar plot B) shows the change in AS population upon detection of aD in the saliva sample.

FIG. 11 illustrates the detection system when several digoxigenin (DIG) molecules act as binding moieties and anti-digoxigenin (aD) is the analyte. Figure A) shows specific sequences comprising modification of a first oligonucleotide 1 (SEQ ID NO:7), a second oligonucleotide 3 (SEQ ID NO: 9) and a third oligonucleotide 5 (SEQ ID NO: 6) according to the invention. The figure also illustrates several DIG moieties for binding of aD. Bar plot B) shows the change in AS population upon detection of aD.

FIG. 12 illustrates the detection system when acting as a competitive/inhibitive sensor. Here digoxigenin (DIG) together with anti-digoxigenin (aD) acts as the binding moiety and free DIG acts as the analyte. Figure A) numbering is the same as FIG. 8A. Bar plots B) show the AS population change in inhibitive assays detecting free DIG in buffer, human plasma and human saliva.

FIG. 13 illustrates the DIG titration curve by inhibitive assay or competitive assay.

FIG. 14 illustrates the detection system when vitamin D (VD) is the binding moiety and vitamin D-binding protein (DBP) is the analyte. Furthermore it also shows free vitamin D as the target in an inhibitive and a competitive assay. The schematic shows the strand displacement of B aided by the binding of DBP onto VD on B. The band shift assay shows a fluorescence image of a native polyacrylamide gel, where only oligonucleotide A is visible. An increase in the AS population is seen upon addition of DBP. The first graph shows the titration of DBP to the sensor. The second and third graphs show the inhibitive and competitive detection of VD respectively.

FIG. 15 shows the design and result of a three-strand aptamer system for DNA detection. Specific sequences comprising modification of the first oligonucleotide 1 (SEQ ID NO: 10), the second oligonucleotide 3 (SEQ ID NO: 11) and the third oligonucleotide 5 (SEQ ID NO: 12) according to the invention are presented. Numbering as indicated in FIGS. 1 and 3. (A) The scheme of ATP detection by structure-switching aptamer. The binding of ATP will shorten the effective toehold on B, thereby hindering the formation of BS. (B) The titration curve of ATP detection.

FIG. 16 shows the system when a split DNA peroxidase signalling system is used. Specific sequences comprising modification of the first oligonucleotide 1 (SEQ ID NO: 13), the second oligonucleotide 3 (one biotin: SEQ ID NO: 5 and two biotins SEQ ID NO: 14) and the third oligonucleotide 5 (SEQ ID NO: 15) according to the invention are presented. Numbering as indicated in FIGS. 1 and 3. With the two-biotin system even visual inspection is possible. Detection with both one biotin and two biotins are shown. (A) The scheme and results of STV detection by using a split DNA peroxidase signalling system. When incorporating two biotins on B, the color difference is obvious in the presence of STV, even distinguishable by with the naked eye. (B) The absorbance of the samples with or without STV, with either one or two biotins.

FIG. 17 shows the design of a one strand system according to the present invention. Numbering as indicated in FIGS. 1 and 3. In addition 11 indicates linker regions.

FIG. 18 presents the one strand system for STV detection and the obtained results when using the sequence presented in example 12. (a) The scheme of STV detection by using a one-strand system. The toehold exchange reaction occurs intramolecularly, and the resulting configuration can be identified by color change from split DNA peroxidase. (b) The absorbance of the one-strand samples with or without STV.

FIG. 19 presents a strategy for detection of special small molecules or ions. (a) The scheme of melamine detection, by making use of the bifacial melamine-thymine recognition through hydrogen-bonding (inset). Without melamine, strand A has longer toehold than strand B, thus having higher priority to bind with S. With melamine, which can serve as a connector for the T-T mismatch, strand B has a longer toehold than A, therefore more BS duplex is expected. (b) Using the same setup as (a), mercury bivalent cation can be detected since Hg²⁺ is well-known for its formation of highly specific “sandwich” complexes at T-T mismatch sites in DNA, in which a Hg²⁺ is bonded linearly between the N1 nitrogens of thymine residues on either side (inset).

FIG. 20 illustrates a more advanced strategy, where strand S has one (a) or two (b) hairpins inside. The function of the internal hairpins is to construct a multi-arm junction around the position of labelled ligand and its binding protein, with the aim of generating larger steric hindrance by its three-dimensional configuration to prevent the hybridization between S and B with a bound protein.

The present invention will now be described in more detail in the following.

### DETAILED DESCRIPTION OF THE INVENTION

Analyte Detection System

In one aspect the present invention relates to an analyte detection system which is based on hybridization equilibriums between three oligonucleotides and is capable of detection of non-nucleic acid analytes in a sample.

An embodiment of this aspect of the invention is an analyte detection system comprising a first oligonucleotide (1), a second oligonucleotide (3), and a third oligonucleotide (5); wherein:
the first or second oligonucleotide comprises a first group (2) forming a first part of a signaling system;
the third nucleotide comprises a second group (6) forming a second part of the signaling system;
at least one covalently linked binding moiety (4) is positioned on the first or second oligonucleotide;
wherein hybridization between the first or second oligonucleotide and the third oligonucleotide generates a signal or is able to catalyze generation of a signal different from when said first or second oligonucleotide and the third oligonucleotide are not hybridized;
and wherein the presence of the analyte changes the hybridization equilibrium of the detection system resulting in a change in signal.

The analyte detection system may e.g. be one wherein:
the first oligonucleotide (1) comprises
   a first toehold region (8) positioned at the 5′-side of a branch migration region (7);
the second oligonucleotide (3) comprises
   a second toehold region (9) positioned at the 3′-side of a branch migration region (7); and
the third oligonucleotide (5) comprises
   a first toehold region (8′);
   a second toehold region (9′); and
   a branch migration region (7′);

wherein:
the first toehold region (8) in the first oligonucleotide (1) and the first toehold region (8′) in the third oligonucleotide (5) comprise complementary sequences;
the branch migration region (7) in the first oligonucleotide (1) and the branch migration region (7′) in the third oligonucleotide (5) comprise a stretch of complementary nucleotides;
the second toehold region (9) in the second oligonucleotide (3) and the second toehold region (9′) in the third oligonucleotide (5) comprise a stretch of complementary nucleotides; and
the branch migration region (7) in the second oligonucleotide (3) and the branch migration region (7′) in the third oligonucleotide (5) comprise a stretch of complementary nucleotides.

In a particular embodiment, the invention relates to an analyte detection system for detecting analytes different from DNA and RNA, the system comprising
a first oligonucleotide (1) comprising
   a first toehold region (8) positioned at the 5′-side of a branch migration region (7);
   optionally at least one covalently linked binding moiety (4);
   optionally a first group (2), said first group forming a first part of a signaling system:
a second oligonucleotide (3) comprising
   a second toehold region (9) positioned at the 3′-side of a branch migration region (7);
   optionally at least one covalently linked binding moiety (4);
   optionally a first group (2), said first group forming a first part of a signaling system;
a third oligonucleotide (5), comprising
   a first toehold region (8′);
   a second toehold region (9′);
   a branch migration region (7′);
   optionally at least one covalently linked binding moiety (4);
   a second group (6), said second group forming a second part of the signaling system;
with the proviso that the first group (2) forming a first part of a signaling system is comprised in either the first oligonucleotide (1) and/or the second oligonucleotide (3);
with the proviso that at least one covalently linked binding moiety (4) is positioned on the first oligonucleotide (1) and/or the second oligonucleotide (3) and/or the third oligonucleotide (5);
wherein the first toehold region (8) in the first oligonucleotide (1) and the first toehold region (8′) in the third oligonucleotide (5) comprise complementary sequences;
wherein the branch migration region (7) in the first oligonucleotide (1) and the branch migration region (7′) in the third oligonucleotide (5) comprise a stretch of complementary nucleotides;
wherein the branch migration region (7) in the second oligonucleotide (3) and the branch migration region (7′) in the third oligonucleotide (5) comprise a stretch of complementary nucleotides;
wherein the second toehold region (9) in the second oligonucleotide (3) and the second toehold region (9′) in the third oligonucleotide (5) comprise a stretch of complementary nucleotides;
wherein hybridization between the first oligonucleotide (1) and the third oligonucleotide (5) generates a signal or is able to catalyze the generation of a signal different from when the first oligonucleotide (1) and the third oligonucleotide (5) are not hybridized, with the proviso that the first group (2) forming a first part of a signaling system is comprised on the first oligonucleotide (1); or
wherein hybridization between the second oligonucleotide (3) and the third oligonucleotide (5) generates a signal or is able to catalyze the generation of a signal different from the signal generated or catalyzed when the second oligonucleotide (3) and the third oligonucleotide (5) are not hybridized, with the proviso that the first group (2) forming a first part of a signaling system is comprised on the second oligonucleotide (3).

As used herein, the term “detecting” is intended to encompass not only qualitative detection of the presence or absence of an analyte, but also quantification of the amount of an analyte using the invention.

References to “an analyte” as used herein include detection of a single analyte as well as, where applicable, two or more analytes.

Although the oligonucleotides A, B and S will often be on separate nucleotide strands, the invention may also be carried out with two or more of the oligonucleotides being partly or fully connected by covalent bonds.

Further, while the invention is generally described herein for the sake simplicity using three oligonucleotides, generally referred to as A, B or S, it will be clear that the method can be performed using additional oligonucleotides, e.g. an additional oligonucleotide C or two additional oligonucleotides C and D, where such additional nucleotides may be similar to oligonucleotides A and/or B as described herein. Alternatively or additionally, the invention may be performed with one or more further oligonucleotides similar to oligonucleotide S as described herein. By way of example, the invention may be performed with one set of oligonucleotides (A, B, S) for detection of a first analyte together with a second set of oligonucleotides (C, D, S′) for detection of a second analyte.

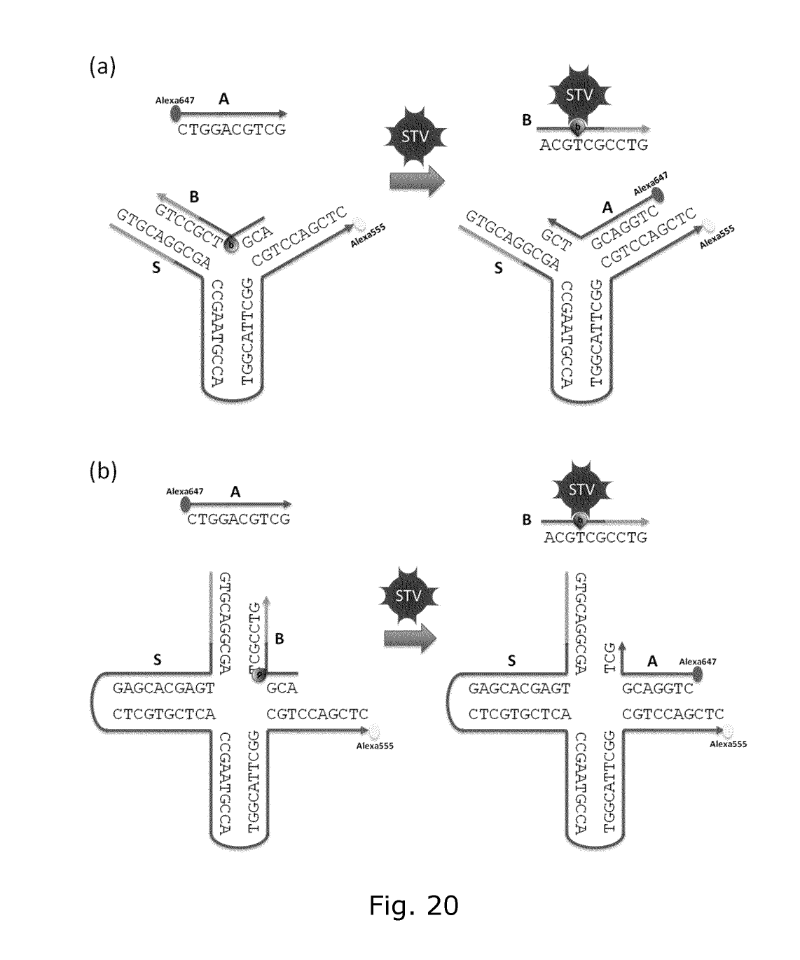

A further alternative is one in which oligonucleotide S comprises more than one hybridization domain. An example of this alternative is illustrated in FIG. 20, where S is shown has having one or more hairpin turns, thereby forming multiple hybridization domains. While the multiple hybridization domains are shown in FIG. 20 as being part of a single oligonucleotide S, it would also be possible to use an arrangement in which two or more such binding domains are located on separate nucleotide strands.

As previously mentioned the system is based on changes in hybridization equilibriums between the oligonucleotides in the system. Thus, in an embodiment the presence of the analyte changes the hybridization equilibrium of the detection system resulting in a change in signal, e.g. compared to when the analyte is absent. Thus, the invention presents a unique system which takes advantage of hybridization events taking place between oligonucleotides (e.g. DNA) to detect the presence or level of non-DNA (or non-RNA) in a sample. This assay has several advantages compared to e.g. ELISA testing.
Very little “hands-on” work is required, making the assay fast and cheap.
The components are stable for a long time.
The assay may be performed under isothermal conditions, making the required equipment cheap.
Small analytes may be more easily detected since only one binding event to the analyte is required. This is in contrast to e.g. ELISA, where two binding sites on the analyte are normally required.
This assay is homogeneous, thus circumventing immobilization and washing process, as well as the trouble of nonspecific adsorption.
This assay circumvents antibody labeling or protein modification.

First Oligonucleotide

The first oligonucleotide (1) forms part of the detection system. In an embodiment the length of the first oligonucleotide (1) is in the range 8-100 nucleotides, such as 10-100, such as 15-100, such as 20-100, such as 30-100, such as 40-100, such as 50-100, such as 60-100, such as 70-100, such as 80-100, such as 90-100, such as 8-90, such as 8-80, such as 8-70, such as 8-60, such as 8-50, such as 8-40, such as 8-30, such as 8-20, or such as 8-15 nucleotides. In yet another embodiment the first oligonucleotide (1) is selected from the group consisting of SEQ ID NO: 1, 4, 7, 10 and 13. Though specific sequences are provided, the invention is by no means limited to these sequences since different sequence combinations can be selected. This is underlined by the fact that the analytes are detected independent of the sequences. Different sequences may be useful if a multiplex assay is designed to detect more than one analyte in a sample. In the example section results with different sets of oligonucleotides are presented.

In the present context the term “5′-side” refers to a nucleic acid sequence which is located at a position which is 5′ to a particular point or region within the nucleic acid molecule. Similarly the term “3′-side” refers to a nucleic acid sequence which is located at a position which is 3′ to a particular point or region within the nucleic acid molecule.

Second Oligonucleotide

The second oligonucleotide (3) forms part of the detection system. In an embodiment the length of the second oligonucleotide (3) is in the range 8-100 nucleotides, such as 10-100, such as 15-100, such as 20-100, such as 30-100, such as 40-100, such as 50-100, such as 60-100, such as 70-100, such as 80-100, such as 90-100, such as 8-90, such as 8-80, such as 8-70, such as 8-60, such as 8-50, such as 8-40, such as 8-30, such as 8-20, or such as 8-15 nucleotides. In yet another embodiment the second oligonucleotide (3) is selected from the group consisting of SEQ ID NO: 2, 5, 8, 9, 11 and 14. As mentioned above, the invention is by no means limited to these sequences.

Third Oligonucleotide

The third oligonucleotide (5) forms part of the detection system. In an embodiment the length of the third oligonucleotide (5) is in the range 8-100 nucleotides, such as 10-100, such as 15-100, such as 20-100, such as 30-100, such as 40-100, such as 50-100, such as 60-100, such as 70-100, such as 80-100, such as 90-100, such as 8-90, such as 8-80, such as 8-70, such as 8-60, such as 8-50, such as 8-40, such as 8-30, such as 8-20, or such as 8-15 nucleotides. In yet another embodiment the third oligonucleotide (5) is selected from the group consisting of specific SEQ ID NO: 3, 6, 12 and 15. As mentioned above, the invention is by no means limited to these sequences.

Each of the oligonucleotides (1, 3, 5) may comprise both natural and/or unnatural nucleotides. Thus, in an embodiment the oligonucleotides (1,3,5) comprise natural and/or unnatural nucleotides. In yet another embodiment the unnatural nucleotides are selected from the group consisting of PNA, LNA, xylo-LNA-, phosphorothioate-, 2′-methoxy-, 2′-methoxyethoxy-, morpholino- and phosphoramidate-containing molecules or the like. An advantage of the unnatural nucleotides is that they may be more biostable, since they are less degradable by e.g. nucleases. However, the oligonucleotide may also be composed of DNA and/or RNA. Thus, in a further embodiment the oligonucleotides are composed of natural nucleic acids such as DNA or RNA, preferably DNA.

The terms “oligonucleotide”, “nucleic acid”, “nucleic acid molecule” or “nucleic acid sequence” as used herein refer to an oligomer or polymer of ribonucleic acid (RNA) or deoxyribonucleic acid (DNA) or mimics/mimetics thereof. This term includes molecules composed of naturally-occurring nucleobases, sugars and covalent internucleoside (backbone) phosphodiester bond linkages as well as molecules having non-naturally occurring nucleobases, sugars and covalent internucleoside (backbone) linkages which function similarly or combinations thereof. Such modified or substituted nucleic acids are often preferred over native forms because of desirable properties such as, for example, enhanced cellular uptake, enhanced affinity for nucleic acid target and increased stability in the presence of nucleases and other enzymes, and are in the present context described by the terms “nucleic acid analogues” or “nucleic acid mimics”. Preferred examples of nucleic acid mimics/mimetics are peptide nucleic acid (PNA-), Locked Nucleic Acid (LNA-), xylo-LNA-, phosphorothioate-, 2′-methoxy-, 2′-methoxyethoxy-, morpholino- and phosphoramidate-containing molecules or the like.

The nucleic acid, nucleic acid molecule or nucleic acid sequence may, for instance, be composed entirely of deoxyribonucleotides, entirely of ribonucleotides, entirely of nucleic acid mimics or analogues or chimeric mixtures thereof. The monomers are typically linked by internucleotide phosphodiester bond linkages. Nucleic acids typically range in size from a few monomeric units, e.g., 5-40, when they are commonly referred to as oligonucleotides, to several thousands of monomeric units. Whenever a nucleic acid or a nucleic acid sequence is represented, it will be understood that the nucleotides are in 5′ to 3′ order from left to right and that “A” denotes deoxyadenosine, “C” denotes deoxycytidine, “G” denotes deoxyguanosine, and “T” denotes thymidine, unless otherwise noted.

Unnatural Nucleotides

As used herein, “unnatural nucleotides” or “nucleic acid analogue” or “artificial nucleotides” is understood to mean a structural analogue of DNA or RNA, designed to hybridise to complementary nucleic acid sequences (1). Through modification of the internucleotide linkage(s), the sugar, and/or the nucleobase, nucleic acid analogues may attain any or all of the following desired properties: 1) optimised hybridisation specificity or affinity, 2) nuclease resistance, 3) chemical stability, 4) solubility, 5) membrane-permeability, and 6) ease or low cost of synthesis and purification. Examples of nucleic acid analogues include, but are not limited to, peptide nucleic acids (PNA), locked nucleic acids “LNA”, 2′-O-methyl nucleic acids, 2′-fluoro nucleic acids, phosphorothioates, and metal phosphonates.

Toehold Regions

In the present context a “toehold region” relates to either the two complementary oligonucleotide regions able to form Watson-Crick base pairing or a toehold region may refer to the single stranded sequence positioned in the oligonucleotide. Thus, it is to be understood that when using the term “toehold region” in relation to a single stranded oligonucleotide it refers to the single stranded toehold region, whereas when using the term “toehold region” in relation to a double stranded (when two single stranded toehold regions are hybridized to each other) it refers to the double stranded region. In the present context each complementary sequence of a double stranded toehold region may be identified as X and X′.

The toehold regions may have different positions. In an embodiment the first (single stranded) toehold region (8′) and the (single stranded) second toehold region (9′) in the third oligonucleotide (5) are located on opposite sides of the branch migration region (7′). Positioning the single stranded toehold regions 8′ and 9′ on opposite sides of the branch migration regions may speed up the assay.

To minimize cross-hybridization between the different toehold regions, the two toehold regions [8, 8′] and [9,9′] may have different sequences. Thus, in an embodiment the first toehold region (7) in the first oligonucleotide (1) and the second toehold region (8) in the second oligonucleotide (1) are dissimilar. In the present context the wording dissimilar may also be understood as having different sequence and therefore are not 100% identical.

The two single stranded toehold regions may also vary in length from each other. In this way a shift in hybridization may be more easily recognized. Thus, in another embodiment the length of the first toehold region (8) is in the range 1-10 nucleotides, such as 1-8 nucleotides, such as 1-6 nucleotides, such as 1-4, nucleotides, such as 2-10 nucleotides, such as 3-10 nucleotides, such as 4-10 nucleotides, such as 5-10 nucleotides, such as 7-10 nucleotide, such as 3 nucleotides, such as 4 nucleotides, such as 5 nucleotides, or such as 6 nucleotides.

As used herein, the term “hybridization” or “annealing” refers to the association of single stranded nucleotides to form a double stranded structure such that a nucleotide in one strand of the double stranded structure undergoes specific Watson-Crick base pairing with a nucleotide on the opposite strand. The term also comprises the pairing of nucleoside analogues, such as deoxyinosine, nucleosides with 2-aminopurine bases, and the like, that may be incorporated into oligonucleotides according to the invention.

In a further embodiment the first toehold region (8) in the first oligonucleotide (1) and the first toehold region (8′) in the third oligonucleotide (5) comprise a stretch of 1-10 complementary nucleotides, such as 2-10, such as 3-10, such as 4-10, such as 5-10, such as 2-8, such as 2-7, such as 2-6, such as 2-5, such as 2-4.

In yet another embodiment the first toehold region (8) in the first oligonucleotide (1) and the first toehold region (8′) in the third oligonucleotide (5) are at least 70% complementary, such as 70-100% complementary, such as 75-100% complementary, such as 80-100% complementary, such as 85-100% complementary, such as 90-100% complementary, such as 95-100% complementary, such as 97-100% complementary, such as 99-100% complementary, or such as 100% complementary.

In another embodiment the length of the second toehold region (9, 9′) is in the range 1-10 nucleotides, such as 1-8 nucleotides, such as 1-6 nucleotides, such as 1-4, nucleotides, such as 2-10 nucleotides, such as 3-10 nucleotides, such as 4-10 nucleotides, such as 5-10 nucleotides, such as 7-10 nucleotide, such as 3 nucleotides, such as 4 nucleotides, such as 5 nucleotides, or such as 6 nucleotides.

In an embodiment the second toehold region (9) in the second oligonucleotide (3) and the second toehold region (9′) in the third oligonucleotide (5) comprise a stretch of 1-10 complementary nucleotides, such as 2-10, such as 3-10, such as 4-10, such as 5-10, such as 2-8, such as 2-7, such as 2-6, such as 2-5, such as 2-4.

Branch Migration Region

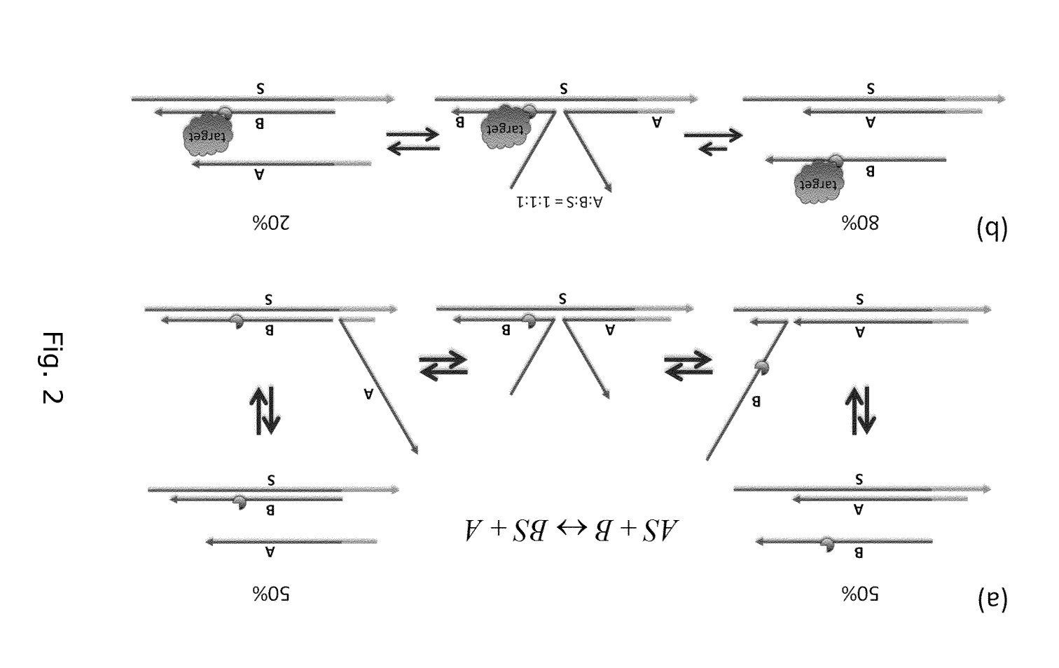

In the present context “branch migration” relates to the situation wherein the equilibrium of the hybridization between a first and a third oligonucleotide are shifted towards hybridization between a second and the third oligonucleotide and vice versa through hybridization in the two toehold regions and the branch migration region. FIG. 2 illustrates branch migration and how the equilibrium situation changes upon binding of an analyte.

The branch migration region facilitates the branch migration between the first and the third oligonucleotide and between the second and the third oligonucleotides, due to the complementary region. Thus, in an embodiment the length of the branch migration region (7, 7′) is in the range 3-30 nucleotides, such as 4-30, such as 5-30, such as 7-30, such as 9-30, such as 11-30, such as 15-30, such as 20-30, such as 25-30, such as 3-25, such as 3-20, such as 3-15, such as 3-11, such as 3-9, such as 3-7, such as 3-5, or such as 3-4 nucleotides. The desired length may be adapted to different temperatures and specific sequences. In yet another embodiment the branch migration region (7) in the second oligonucleotide (3) and the branch migration region (7′) in the third oligonucleotide (5) comprise a stretch of 3-30 complementary nucleotides, such as 4-30, such as 5-30, such as 7-30, such as 9-30, such as 11-30, such as 15-30, such as 20-30, such as 25-30, such as 3-25, such as 3-20, such as 3-15, such as 3-11, such as 3-9, such as 3-7, such as 3-5, or such as 3-4 complementary nucleotides.

In a further embodiment the branch migration region (7) in the second oligonucleotide (3) and the branch migration region (7′) in the third oligonucleotide (5) are at least 70% complementary, such as 70-100% complementary, such as 75-100% complementary, such as 80-100% complementary, such as 85-100% complementary, such as 90-100% complementary, such as 95-100% complementary, such as 97-100% complementary, such as 99-100% complementary, or such as 100% complementary. In yet a further embodiment the branch migration region (7) in the first oligonucleotide (1) and the branch migration region (7) in the second oligonucleotide (3) comprise a stretch of 3-30 complementary nucleotides, such as 4-30, such as 5-30, such as 7-30, such as 9-30, such as 11-30, such as 15-30, such as 20-30, such as 25-30, such as 3-25, such as 3-20, such as 3-15, such as 3-11, such as 3-9, such as 3-7, such as 3-5, or such as 3-4 complementary nucleotides.

In another embodiment the branch migration region (7) in the first oligonucleotide (1) and the branch migration region (7) in the second oligonucleotide (3) are at least 70% identical, such as 70-100% identical, such as 75-100% identical, such as 80-100% identical, such as 85-100% identical, such as 90-100% identical, such as 95-100% identical, such as 97-100% identical, such as 99-100% identical, or such as 100% identical. Thus, the two branch migration regions 7 in the first and the second oligonucleotides do not need to be completely identical.

In an additional embodiment the branch migration region (7) in the first oligonucleotide (1) and the branch migration region (7′) in the third oligonucleotide (5) comprise a stretch of 3-30 complementary nucleotides, such as 4-30, such as 5-30, such as 7-30, such as 9-30, such as 11-30, such as 15-30, such as 20-30, such as 25-30, such as 3-25, such as 3-20, such as 3-15, such as 3-11, such as 3-9, such as 3-7, such as 3-5, or such as 3-4 complementary nucleotides. In a further embodiment the first oligonucleotide (1) and the branch migration region (7′) in the third oligonucleotide (5) are at least 70% complementary, such as 70-100% complementary, such as 75-100% complementary, such as 80-100% complementary, such as 85-100% complementary, such as 90-100% complementary, such as 95-100% complementary, such as 97-100% complementary, such as 99-100% complementary, or such as 100% complementary.

The term ‘sequence identity’ indicates a quantitative measure of the degree of homology between two amino acid sequences or between two nucleic acid sequences of equal length. If the two sequences to be compared are not of equal length they must be aligned to give the best possible fit, allowing the insertion of gaps or, alternatively, truncation at the ends of the polypeptide sequences or nucleotide sequences. The sequence identity can be calculated as (N_{ref}−N_{dif})¹⁰⁰/N_{ref}, wherein N_{dif} is the total number of non-identical residues in the two sequences when aligned and wherein N_{ref} is the number of residues in one of the sequences. Hence, the DNA sequence AGTCAGTC will have a sequence identity of 75% with the sequence AATCAATC (N_{dif}=2 and N_{ref}=8). A gap is counted as non-identity of the specific residue(s), i.e. the DNA sequence AGTGTC will have a sequence identity of 75% with the DNA sequence AGTCAGTC (N_{dif}=2 and N_{ref}=8).

In all polypeptide or amino acid based embodiments of the invention the percentage of sequence identity between one or more sequences is based on alignment of the respective sequences as performed by clustalW software (www.ebi.ac.uk/clustalW/index.html) using the default settings of the program. With respect to the nucleotide-based embodiments of the invention, the percentage of sequence identity between one or more sequences is also based on alignments using the clustalW software with default settings. E.g. for nucleotide sequence alignments these settings are: Alignment=3Dfull, Gap Open 10.00, Gap Ext. 0.20, Gap separation Dist. 4, DNA weight matrix: identity (IUB).

It is to be understood that the degree of complementary can be calculated in a similar way, e.g. by using the complementary sequence of one of the oligonucleotides.

In an embodiment, hybridization between the branch migration region 7 and the branch migration region 7′ comprises one or more hairpins in the branch migration region, such as 1-5 hairpins, such as 1-3 hairpins, such as 1-2 hairpins or such as 1 hairpin. Without being bound by theory it is hypothesized that with two neighboring hairpins in the middle of S, the whole complex of AS will have a configuration of a holiday junction, which will expand a particular 3D structure in the presence of Mg²⁺, thus probably resulting in bigger steric hindrance for the protein-bound B strand to compete with A to hybridize with S (FIG. 20). In an embodiment the one or more hairpins have a length of 1-20 nucleotides, such as 3-20, such as 5-20 nucleotides, such as 10-20 nucleotides, such as 3-15 nucleotides, such as 3-10 nucleotides, or such as 5-15 nucleotides.

Binding Moiety

The binding moiety (or moieties) according to the present invention allows the assay to detect analytes which do not form Watson-Crick base pairing. Similarly it allows for detection of analytes which do not bind to DNA or RNA, such as DNA binding proteins.

The one or more binding moieties may in principle be located on any of the three oligonucleotides. In an embodiment the at least one covalently linked binding moiety (4) is positioned on the first oligonucleotide (1). In another embodiment the at least one covalently linked binding moiety (4) is positioned on the second oligonucleotide (3). In a third embodiment the at least one covalently linked binding moiety (4) is positioned on the third oligonucleotide (5). In an embodiment the binding detection system comprises 1-5 binding moieties, such as 1-4, such as 1-3, such as 1-2, such as 1, such as 2-5, or such as 3-5.

The one or more binding moieties may be positioned at different locations. In an embodiment the at least one covalently linked binding moiety (4) is covalently linked to part of the branch migration region (7) in the first oligonucleotide (1), the second oligonucleotide (3) or the third oligonucleotide (5).

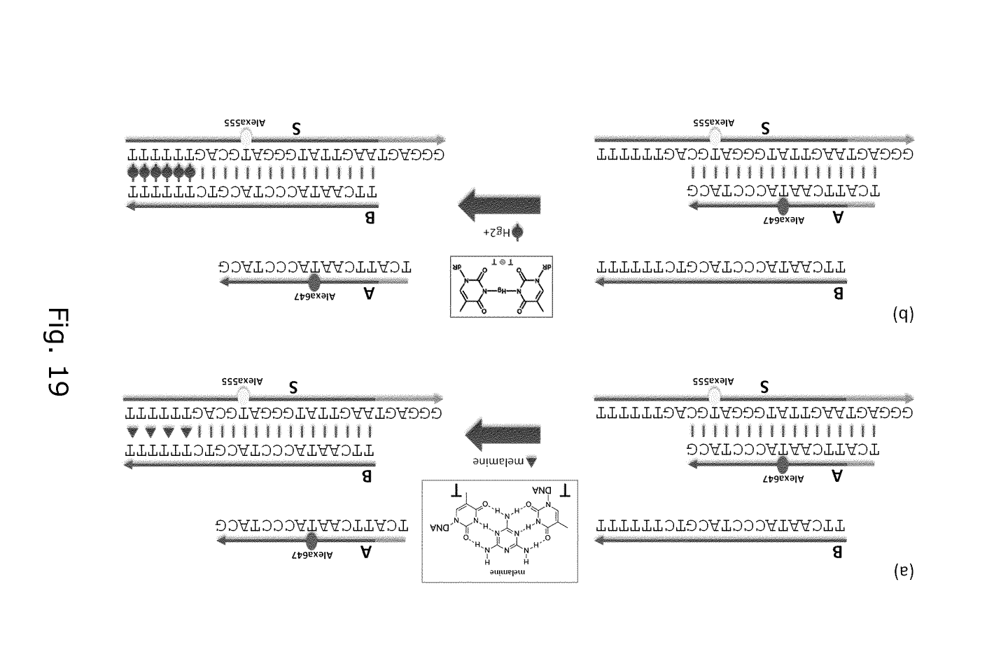

Different types of binding moieties may be employed depending on the specific analyte to be detected. Thus, in an embodiment the at least one covalently linked binding moiety (4) is selected from the group consisting of an organic molecule, an antibody, an antigen, an aptamer, biotin, and a hapten. In another embodiment the antigen is a protein antigen a peptide antigen, or a sugar antigen. In some cases, no covalent conjugation is needed, since some analyte may be able to bind with one or a few nucleotides directly. These analytes include DNA-binding protein, some ions (FIG. 19b) or intercalators, and small molecules such as melamine (FIG. 19a). In an embodiment the binding moiety may also have a sandwich structure, in the sense that a covalently linked first moiety functions as a handle for a second binding moiety which can be bound covalently or non-covalently to the first moiety. Thus, a second binding moiety may have a dual function I) binding to the first moiety and II) binding site for the analyte. In the case of e.g. an aptamer that forms part of one or more of the oligonucleotides and that is capable of binding to the analyte, no separate covalently linked binding moiety may be required.

Small organic molecules are preferred binding moieties. In an embodiment the organic molecule has a molecular weight in the range 150-1500 Da (Dalton), such as 150-1200 Da, such as 150-1000 Da, such as 150-800 Da, such as 150-600 Da, such as 150-400 Da, such as 150-300 Da, such as 300-1500 Da, such as 400-1500 Da, such as 600-1500 Da, such as 800-1500 Da, such as 1000-1500 Da, or such as 1200-1500 Da. Small organic molecules are also preferred analytes for the present invention. In a more specific embodiment the at least one covalently linked binding moiety (4) is selected from the group consisting vitamin D, folate, enrofloxacin, digoxigenin. Further examples of small organic molecules and/or covalenty linked binding moieties according to the present invention are:

Toxins: Staphylococcal enterotoxin B (SEB), Staphylococcal enterotoxin A (SEA), Domoic acid (DA), Aflatoxin (AFB1, AFG1, AFB2, AFG2, AFM1), deoxynivalenol, ochratoxin A (OTA).

Drugs: morphine-3-glucuronide (M3G), oral anticoagulant warfarin, insulin.

Pesticides: atrazine, simazine, chlorpyrifos, carbaryl, dichlorodiphenyltrichloroethane (DDT), 2,4-dichlorophenoxyacetic acid (2,4-D).

Other Environmental Analytes: 2-hydroxybiphenyl (HBP), benzo[a]pyrene (BaP). Phenols (bisphenol A, Atrazine, polychlorinated biphenyls,3,7,8-TCDD), melamine and related compounds.

Veterinary Drugs/antibiotics: penicillins and cephalosporins, Chloramphenicol and chloramphenicol glucuronide, fenicol antibiotic residues, tetracycline, tylosin, erythromycin, sulfonamide antibiotics.

Chemical Contaminants: 4-nonylphenol in shellfish, insulin-like growth factor-1 (IGF-1) in cows.

Vitamins: Vitamin B2 (riboflavin), vitamin B5 (pantothenic acid), vitamin B8 (biotin), vitamin B9 (folic acid), vitamin B12 (cobalamine).

Hormones: progesterone, human chorionic gonadotropin hormone (hCG), 17β-estradiol, R-fetoprotein (AFP), testosterone, 19-nortestosterone, methyltestosterone, boldenone and methylboldenone.

Explosives: 2,4,6-Trinitrotoluene (TNT), 2,4,6-trinitrophenol (TNP), 1,3,5-Trinitrobenzene (TNB), triacetonetriperoxide (TATP), hexamethylene triperoxide diamine (HMTD), pentaerythritol tetranitrate (PETN), cyclotrimethylenetrinitramine (RDX).

It is to be understood that the above list of compounds may also be the analytes to be detected according to the present invention.

Antibody and Other Protein Analytes:

Diagnostic Antibodies: Mycoplasma hyopneumoniae antibody, Classical swine fever virus (CSFV) antibody.

Antibodies against Viral Pathogens: antibodies against hepatitis G, antibodies against human hepatitis B virus (hHBV), antibodies against herpes simplex virus type 1 and type 2 (HSV-1, HSV-2), antibodies against Epstein-Barr virus (anti-EBNA), Antibodies against human respiratory syncytial virus (RSV), anti-adenoviral antibodies.

Drug-Induced Antibodies: antibodies against insulin orgranulocyte macrophage colony stimulating factor (GM-CSF), antibodies against other recombinant or non-recombinant protein or antibody drugs.

Proteins: caseins, immunoglobulin G, folate-binding protein, lactoferrin, and lactoperoxidase.

Allergens or Allergy Markers: peanut allergens, Conalbumin/Tropomyosin in pasta, Sesame seed protein, tropomyosin, immunoglobulin E (IgE) antibody, histamine (â-imidazole ethylamine.

Cancer Markers: prostate-specific antigen (PSA), PSA-ACT complex (α1-antichymotrypsin), carbohydrate antigen (CA 19-9), Protein vascular endothelial growth factor (VEGF), interleukin-8 (IL-8), Carcinoembryonic antigen (CEA), fibronectin.

Other makers: troponin (cTn I), antibodies against glucose 6-phosphate isomerase (GPI), anti-glutamic acid decarboxylase (GAD) antibodies, c-reactive protein (CRP), cystatin C, hepatitis B surface antigen (HBsAg).

The binding moiety may have its binding partner bound to the binding moiety. In the present context it is to be understood that the term “binding partner” refers to a molecule which may bind non-covalenty to the binding moiety. Thus, in an embodiment the binding partner is bound non-covalently to the binding moiety. Non-limiting examples of “binding moiety”-“binding partner” couples are antibody-antigen, streptavidin-biotin, Folate receptor-folate. Thus, in an embodiment the binding moiety has its binding partner bound to the binding moiety.

Three strategies may be adopted for the present detection system, depending in part on the type of analyte being detected. These strategies are outlined below, exemplified with reference to detection of for example a protein or antibody (Strategy 1) or a small molecule analyte (Strategies 2 and 3).

Strategy 1: Direct Assay

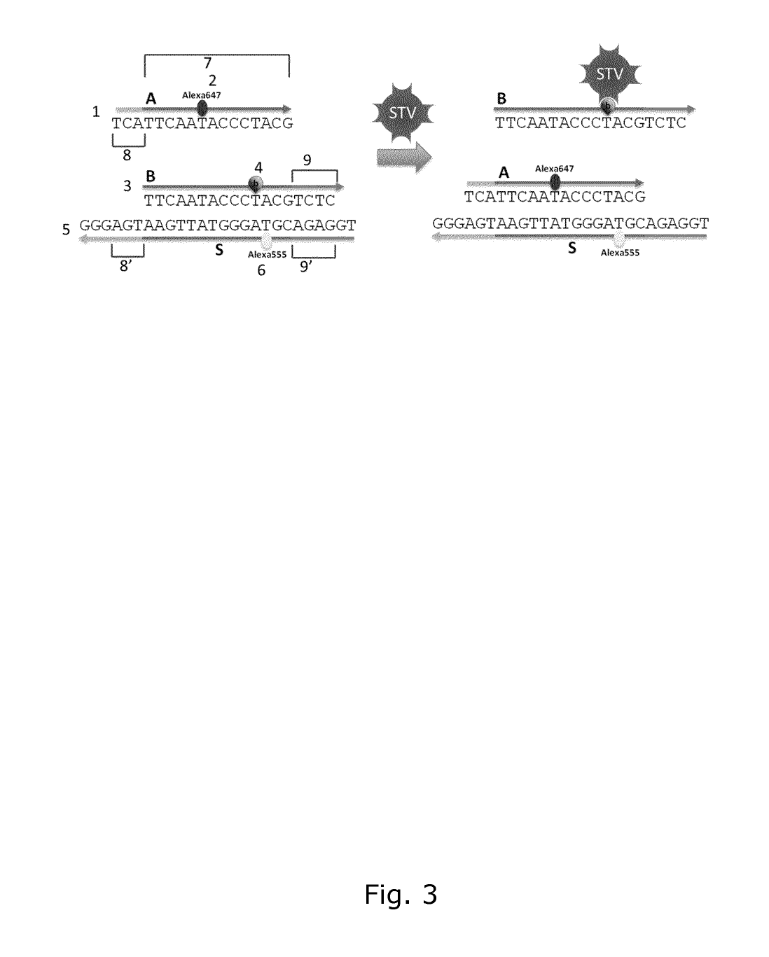

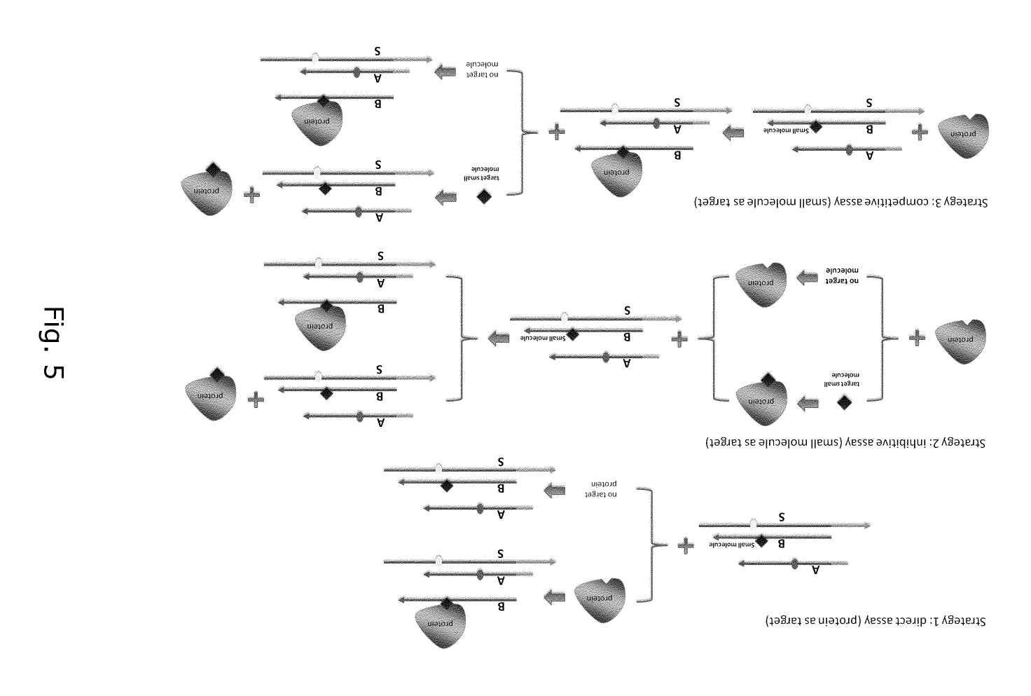

In one embodiment (strategy 1) a protein or antibody is the analyte to be detected (FIG. 5, strategy 1). Using this strategy binding of the protein or antibody (analyte) to the binding moiety shifts the hybridization equilibrium, which can then subsequently be detected. This strategy is outlined in further detail in FIGS. 3-5 and the corresponding examples 1 and 2.

Strategy 2: Inhibitive Assay

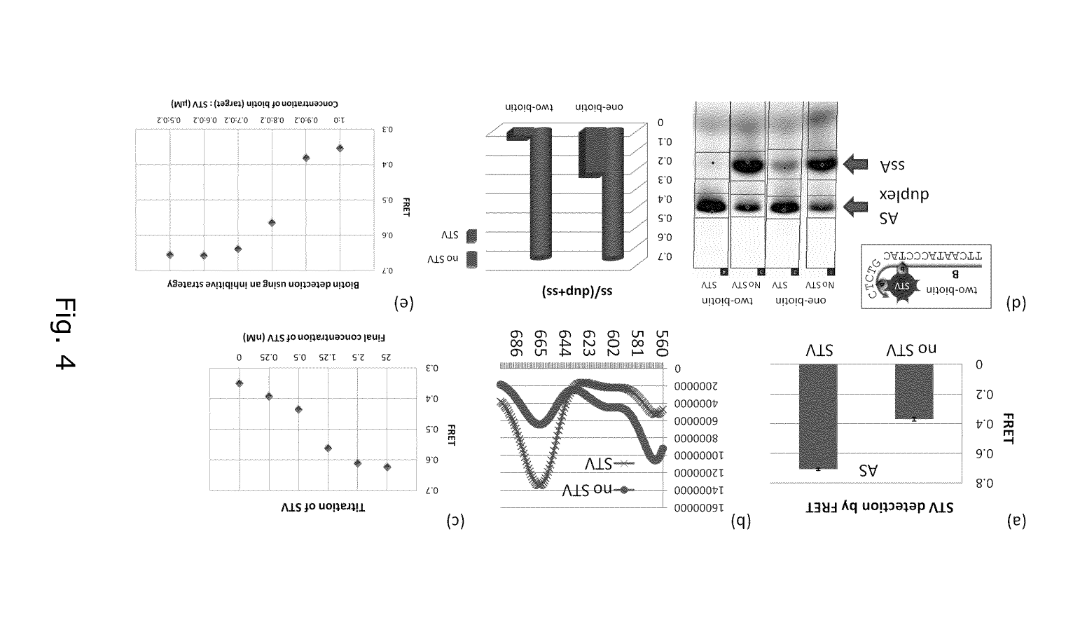

In another embodiment (strategy 2), a sample comprising (or suspected of comprising) a small molecule analyte (target molecule) is first mixed with the binding partner (e.g. protein such as an antibody) to the binding moiety, then the mixture is added into the remaining part of the detection system (FIG. 5, strategy 2). If there are target small molecules (analytes) included in the sample, they will take up the binding sites of the protein (binding partner), thus resulting in little or no binding ability of the protein to interfere with the hybridization equilibrium through interaction with the same covalently linked small molecule linked on oligonucleotide B. Thus, the presence of analyte in a sample will result in a change in hybridization equilibrium between the oligonucleotides of the detection system. This is called an inhibitive assay. FIG. 4e shows the result of such an assay for biotin detection.

Strategy 3: Competitive Assay

In yet another embodiment (strategy 3) the binding partner (protein) is first added into the assay and causes the same equilibrium shift as strategy 1, followed by adding the unknown sample suspected of comprising the analyte (target molecule).

If there are free analyte molecules present, they may replace the covalently linked binding moiety which binds to the binding partner (protein) on oligonucleotide B to bind with protein, thus changing the hybridization equilibrium (FIG. 5, strategy 3). Thus, this strategy is a competitive assay. In both the inhibitive and competitive strategies, the bigger the signal changes compared to the original assay, the fewer target molecule existing in the sample.

Analytes

Different kinds of analytes may be detected by the present invention. As previously mentioned the analytes are not DNA or RNA. Similarly the analyte may not be a DNA interacting analyte such as a DNA or RNA binding protein. Examples of analytes which do not form Watson-Crick base pairing but do bind to DNA are DNA binding proteins such as histones. In an embodiment the analyte is a non-DNA and non-RNA binding analyte.

In an embodiment the analyte is selected from the group consisting of proteins, peptides, organic molecules, antibodies, antigens, sugars, lipids and haptens. In another embodiment analyte is a protein antigen, a peptid antigen, or a sugar antigen. In yet another embodiment the analyte is an organic molecule having a molecular weight in the range 150-1500 Da, such as 150-1200 Da, such as 150-1000 Da, such as 150-800 Da, such as 150-600 Da, such as 150-400 Da, such as 150-300 Da, such as 300-1500 Da, such as 400-1500 Da, such as 600-1500 Da, such as 800-1500 Da, such as 1000-1500 Da, or such as 1200-1500 Da. In a specific embodiment the analyte is selected from the group consisting vitamins, toxins, allergens, explosives, drugs, such as cocaine, antibiotics such as enrofloxacin, pesticides, hormones, chemical contaminants, biomarkers. Further, above is a more extensive list of analytes according to the present invention.

Signaling System

The detecting system according to the present invention comprises a signaling system providing a signal or change in signal when an analyte is detected. The signaling system is based on the principle that a signal is generated or catalyzed when oligonucleotides are hybridized which comprises the different parts of the signaling system. As illustrated in e.g. example 1, hybridization of the oligonucleotide 1 (A) and 5 (S) generates an increased signal when the two parts of the signaling system are brought into proximity, whereas hybridization of oligonucleotide 3 and 5 does not result in the generation of a signal. Thus, in an embodiment the second oligonucleotide (3) comprises the first group (2), said first group forming a first part of a signaling system and the third oligonucleotide (5) comprises the second group (6), said second group forming a second part of a signaling system.

Similarly the signaling system may be divided between the first and the third oligonucleotide. Thus, in an embodiment the first oligonucleotide (1) comprises the first group (2), said first group forming a first part of a signaling system and the third oligonucleotide (5) comprises the second group (6), said second group forming a second part of a signaling system.

The signaling system may also comprise a third part providing different signals depending on which oligonucleotides are hybridized to each other, meaning that each oligonucleotide comprises a part of the detection system. Thus, in an embodiment
the first oligonucleotide (1) comprises the first group (2), said first group forming a first part of a signaling system;
the second oligonucleotide (3) comprises a third group (10), said third group (10) forming a third part of the signaling system; and
the third oligonucleotide (5) comprises the second group (6), said second group forming a second part of a signaling system.

This setup is illustrated in FIG. 4 and the corresponding example.

Different types of signaling systems may be employed. Thus, in an embodiment the signaling system formed by the first group (2) and the second group (6) is a quencher-fluorophore signaling system, a fluorophore-quencher signaling system, a FRET signaling system, a DNA peroxidase catalysis signaling system, or a quencher and singlet oxygen sensitizer. The signaling system may employ fluorescent nanoparticles, in particular in the case of a FRET or quencher system. The example section provides different examples of setups which may be employed according to the present invention. In yet another embodiment
I. the first group (2) forming a first part of a signaling system is a quencher and the second group (6) forming a second part of a signaling system is a fluorophore, or
II. the first group (2) forming a first part of a signaling system is a fluorophore and the second group (6) forming a second part of a signaling system is a quencher, or
III. the first group (2) forming a first part of a signaling system is a FRET donor and the second group (6) forming a second part of a signaling system is a FRET acceptor, or
IV. the first group (2) forming a first part of a signaling system is a FRET acceptor and the second group (6) forming a second part of a signaling system is a FRET donor, or
V. the first group (2) forming a first part of a signaling system is a first half of a DNA peroxidase signaling system and the second group (6) forming a second half of the DNA peroxidase signaling system.

When using the DNA peroxidase signaling system the detection system may comprise further components. Thus, in a further embodiment the detection system comprises hemin and/or ABTS²⁻ and/or H₂O₂ and/or luminol. Colorimetric methods are also possible to integrate into the assays according to the present invention, such as gold nanoparticles (AuNP) or quantum dots (QD).

The different parts of the signaling system may be covalently linked at different locations on the oligonucleotides. Thus, in an embodiment the first part of the signaling system and/or the second part of the signaling system and/or the third part of the signaling system is covalently linked to part of the branch migration region (7, 7′) on the oligonucleotide to which it is coupled.

In another embodiment the first part of the signaling system and/or the second part of the signaling system and/or the third part of the signaling system is covalently linked to part of the toehold region (7, 8) on the oligonucleotide to which it is coupled.

The person skilled in the art knows how to design e.g. FRET pairs so that a signal is generated or increased when the FRET pair comes into proximity. Similarly the skilled person knows how to design a fluorophore-quencher pair so that a signal disappears or weakens when the fluorophore-quencher pair comes into proximity. In Example 3 and the corresponding figures it is shown how a DNA peroxidase assay may be designed to catalyze the generation of a signal when each of the oligonucleotides comprising a part of the DNA peroxidase signaling system are brought into proximity. One advantage of the DNA peroxidase signaling system is that it constitutes an amplification reaction, which may allow for detection of very small amount of analyte. Another advantage is that the oligonucleotides are more easily synthesized since fewer modifications are required. In all the above examples the different pairs are brought into proximity due to hybridization of the two oligonucleotides comprising each part of the signaling system. As described above the assay is based on an equilibrium reaction, thus, the signal may be strengthen or weakened upon a change in the hybridization equilibrium, e.g. due to the binding or release of a binding partner to the covalently linked binding moiety.

In a special embodiment the analyte may be a nucleic acid molecule such as DNA or RNA, with the proviso that the signaling system is a DNA peroxidase signaling system.

In another special embodiment the analyte may be melamine and structurally related compounds that can interact with one, two or three thymine bases in either of the oligonucleotides (1), (3) and/or (5)

Covalently Linked Oligonucleotides

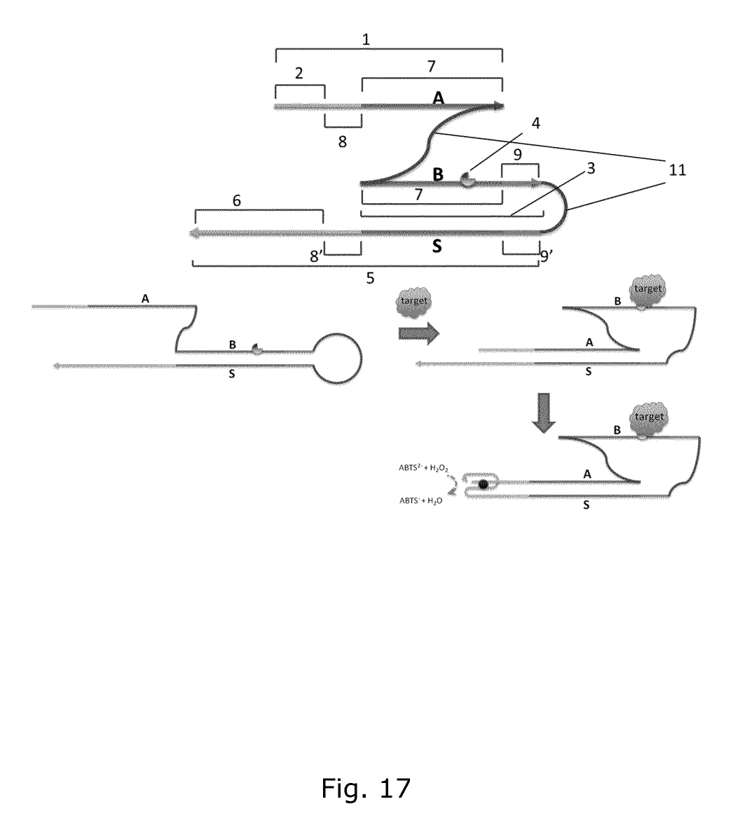

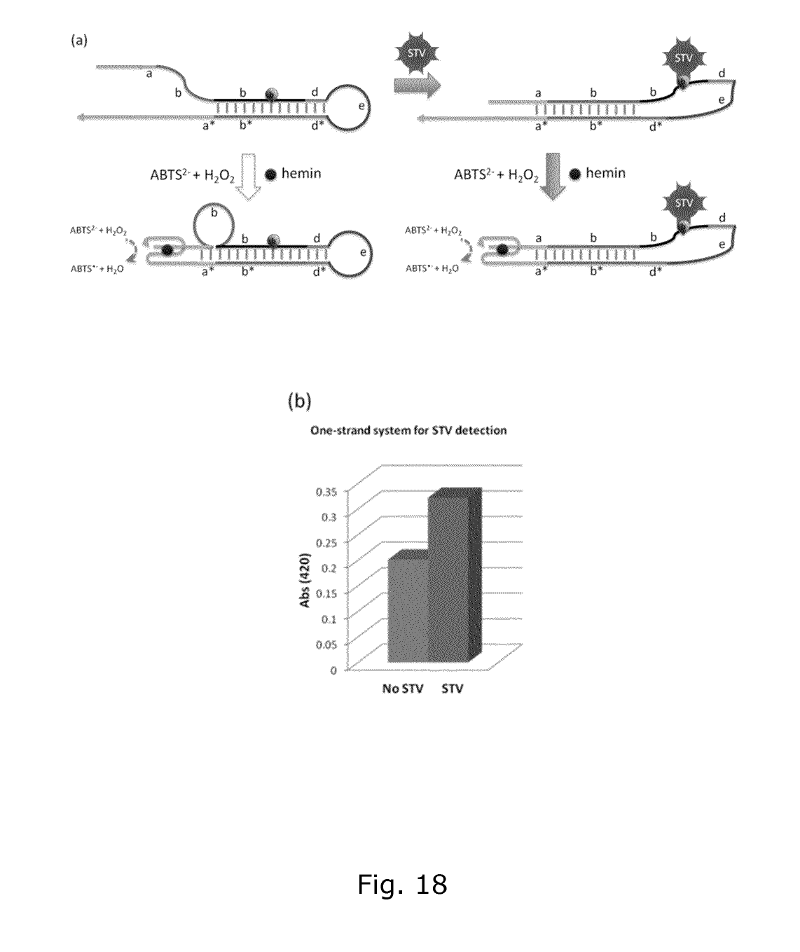

In some cases it may be a disadvantage that the detection system is composed of several oligonucleotides, since it may increase the time before a hybridization equilibrium is reached. The oligonucleotides according to the present invention may also be covalently linked to each other, meaning the detection system is only composed of one or two individual oligonucleotides instead of being made of three individual oligonucleotides. This principle is illustrated in example 12 and the corresponding FIGS. 17 and 18. Thus, in an embodiment the first oligonucleotide, the second oligonucleotide and the third oligonucleotide are covalently linked.

Thus, in an embodiment said first oligonucleotide (1) is covalently linked to the second oligonucleotide (3). In another embodiment the third oligonucleotide (5) is covalently linked to the first oligonucleotide (1). In a further embodiment the second oligonucleotide (3) is covalently linked to the third oligonucleotide (5). In a further embodiment said first oligonucleotide (1) is covalently linked to the second oligonucleotide (3) and second oligonucleotide (3) is covalently linked to the third oligonucleotide. In yet another embodiment the 3′-end of the first oligonucleotide (1) is covalently linked to the 5′-end of the second oligonucleotide (3). In a further embodiment the 3′-end of the second oligonucleotide (3) is covalently linked to the 5′-end of the third oligonucleotide (5).

It is to be understood that when the two or more of the oligonucleotides are covalently linked to each other, the detection system still comprises the first, second and third oligonucleotides according to the present invention.

In a special embodiment the analyte may be a nucleic acid molecule such as DNA or RNA, with the proviso that two or more of the oligonucleotides are covalently linked as described above.

In yet another embodiment the oligonucleotides are covalently linked by linkers. In an additional embodiment the linkers are selected from the group consisting of phosphodiester bond linkages, nucleotides, such as from 1-20 nucleotides, such as 1-10, 1-5, 3-10, oligonucleotides, peptides, a C-linker, such as a C1-C20 linker, PEG-linkers, disulfide likers, and sulfide linkers. The person skilled in the art may find other suitable linkers.

Kit of Parts

The detection system may be provided in the form of a kit of parts. Thus, an aspect of the present invention relates to a kit of parts comprising an analyte detection system according to the present invention.

In another aspect the invention relates to a kit of parts comprising the first oligonucleotide 1, the second oligonucleotide 3 and the third oligonucleotide 5 according to the present invention.

The kit may comprise further components. Thus, in an embodiment the kit further comprises hemin and/or ABTS²⁻ and/or H₂O₂. These components may be part of the kit when a DNA peroxidase assay is used. As previously described it may be advantageous to include the binding partner to the binding moiety in the kit. Thus, in a further embodiment the kit comprises the binding partner to the binding moiety. In yet another embodiment the binding partner is non-covalently coupled to the one or more binding moieties. In yet another embodiment the binding partner is in a separate compartment of the kit than the oligonucleotide comprising the covalently linked binding moiety.

Method for detection the presence or level of an analyte in a sample The present invention also provides a method for detecting the presence of an analyte in a sample. Thus, an aspect of the present invention relates to a method for detection the presence or level of an analyte different from DNA and RNA in a sample comprising
a) providing a sample suspected of comprising or comprising the analyte of interest;
b) providing the analyte detection system according to the present invention;
c) incubating the sample with the analyte detection system;
d) comparing the detected level of analyte to a reference level; and
e) determining the presence or level of analyte in the sample.

It is to be understood that the sample may be known to comprise the sample or it may be unknown whether the analyte is present in the sample. In the first instance quantification may be the goal whereas for the second instance just detecting the presence may be enough.

In yet another embodiment the analyte different from DNA and RNA is not a nucleic acid molecule.

Preferably the incubation step is continued until a hybridisation equilibrium is reached. Thus, in an embodiment the incubation step c) is continued until equilibrium is reached. However, the assay may also be monitored in real time. The hybridisation equilibrium may change upon binding of the analyte to the binding moiety. Thus, in an embodiment the hybridization equilibrium between the first oligonucleotide (1) and the third oligonucleotide (5) and between the second oligonucleotide (3) and the third oligonucleotide (5) is shifted upon binding of an analyte to the binding moiety.

In the case the binding partner to the binding moiety is included in the assay, the hybridization equilibrium is changed in a different way. Thus, in another embodiment the hybridization equilibrium between the first oligonucleotide (1) and the third oligonucleotide (5) and between the second oligonucleotide (3) and the third oligonucleotide (5) is shifted upon release of a binding partner to the binding moiety (4) from the binding moiety.

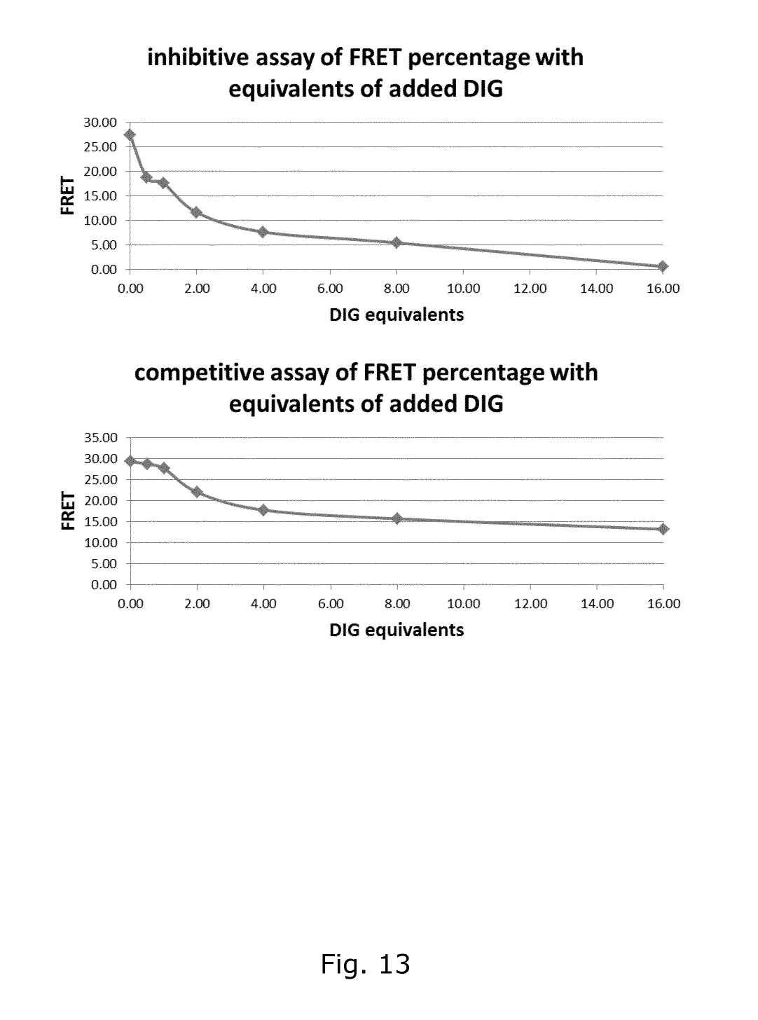

The binding partner to the binding moiety may be included in the assay at different points in time. In yet another embodiment the binding partner to the binding moiety is incubated with the sample before the sample is incubated with the oligonucleotides of the detection system. In another embodiment the binding partner to the binding moiety is incubated with the sample after the sample is incubated with the oligonucleotides of the detection system. In yet another embodiment the binding partner to the binding moiety is incubated with the oligonucleotides of the detection system before incubation with the sample. Depending on the binding affinities each of the above solution may be the most suitable. For further details see also FIG. 13.

In a further embodiment the release of a binding partner from the binding moiety is caused by binding an analyte to the binding partner.

The time of incubation may vary from assay to assay, depending on the sample type, the analyte and the precise oligonucleotides employed. Thus, in an embodiment the incubation step c) takes place for a period of 1 minute-24 hours, such as 1 minute-12 hours, such as 1 minute 6 hours, such as 1 minute to 2 hours, such as 1-60 minutes, such as 1-30 minutes, such as 1-15 minutes such as 1-5 minutes, such as 5-60 minutes, such as 10-60 minutes, such as 15-60 minutes, such as 30-60 minutes, such as 1-6 hours, such as 2-6 hours or such as 4-6 hours.

An advantage of the present assay is that the temperature may not need to be changed during the assay. Thus, in a further embodiment the method is performed under isothermal conditions. This means that the assay be performed using only a heating chamber or heating plate. Similarly the assay may be performed in the field at room temperature. The assay can then subsequently be analysed using the required equipment. In case the DNA peroxidase signalling system is used, the result may be determined by visual inspection. In yet another embodiment the method is performed at a temperature in the range 4-50° C., such as 10-50° C., such as 20-50° C., such as 25-50° C., such as 30-50° C., such as 35-50° C., such as 40-50° C., such as 4-40° C., such as 4-35° C., such as 4-30° C., such as 4-25° C., or such as 4-20° C. In yet another embodiment the assay is performed under isothermal conditions.

Sample

The sample may be from different origins. In an embodiment the sample is a sample obtained from the environment such as a water sample. In another embodiment the sample is a biological sample. In a further embodiment the sample is a food sample, or a plastic. Plastic may be tested for the presence of softeners which may be toxic

In yet another embodiment the biological sample has been obtained from a mammal, such as a human. In a further embodiment the biological sample is a blood sample, such as a serum or plasma, a urine sample, a faeces sample, a biopsy sample, or a saliva sample. To provide the optimal conditions for the assay the sample may be purified or substantially purified before employed in the assay according to the present invention. Thus, in yet another embodiment the sample is a purified sample. An advantage of purifying the sample is that the reaction conditions can be more easily controlled.

The assay may be read by different methods. Thus, in an embodiment the presence or level of analyte is determined by visual inspection, optical density, spectroscopy, absorbance spectroscopy, fluorescent spectroscopy, electrochemistry, QCM, SPR, or microscopy.

To be able to determine the level or presence, the sample may need to be compared to a reference level. Thus, in yet another embodiment the reference level is a predetermined value, a standard curve, or a negative control. The reference level may be set based on different criteria e.g. by the use of a ROC curve which is often used in e.g. diagnostic tests.

The accuracy of a diagnostic test may be characterized by a Receiver Operating Characteristic curve (“ROC curve”). An ROC is a plot of the true positive rate against the false positive rate for the different possible cutoff points of a diagnostic test. An ROC curve shows the relationship between sensitivity and specificity. That is, an increase in sensitivity will be accompanied by a decrease in specificity. The closer the curve follows the left axis and then the top edge of the ROC space, the more accurate the test. Conversely, the closer the curve comes to the 45-degree diagonal of the ROC graph, the less accurate the test. The area under the ROC is a measure of test accuracy. The accuracy of the test depends on how well the test separates the group being tested into those with and without the disease in question. An area under the curve (referred to as “AUC”) of 1 represents a perfect test, while an area of 0.5 represents a less useful test. Thus, biomarker and diagnostic methods of the present invention have an AUC greater than 0.50, more preferred tests have an AUC greater than 0.60, still more preferred tests have an AUC greater than 0.70.

Other useful measures of the utility of a test are positive predictive value and negative predictive value. Positive predictive value is the percentage of people who test positive that are actually positive. Negative predictive value is the percentage of people who test negative that are actually negative. Thus, the skilled person will be able to determine a reference level based on the specific required criteria.

It should be noted that embodiments and features described in the context of one of the aspects of the present invention also apply to the other aspects of the invention. It should also be noted that where reference numerals are used in the present specification and claims to exemplify the invention by reference to the drawings, these are for illustrative purposes only and should not be construed as limiting the invention.

All patent and non-patent references cited in the present application are hereby incorporated by reference in their entirety.

The invention will now be described in further detail in the following non-limiting examples.

### EXAMPLES

### Example 1

Detection of a Protein: Streptavidin (STV)

Materials

Three DNA strands are used in this experiment, named A, B, and S respectively. A and S are with internal amine modification, which is used as a handle for Alexa fluorophore labeling, while B has an internal biotin modification. The sequences are given below (underlining indicates the toehold regions):

| name | sequence | modification |
|---|---|---|
| A- | TCATTCAA1ACCCTACG | 1: Int Amino Modifier C6 |
| SEQ ID NO: 4 | | dT |
| B- | TTCAATACCC2ACGTCTC | 2: Int Biotin dT |
| SEQ ID NO: 5 | | |
| S- | TGGAGACG1AGGGTATTGAATGAGGG | 1: Int Amino Modifier C6 |
| SEQ ID NO: 6 | | dT |

All the oligonucleotides were purchased from DNA Technology A/S in Denmark. RP-HPLC purification was done by the company directly after synthesis.

Alexa Fluor® 647 Succinimidyl Ester and Alexa Fluor® 555 Succinimidyl Ester were purchased from Invitrogen.

All the other reagents, including streptavidin, were purchased from Sigma-Aldrich. FIG. 3 illustrates how the oligonucleotides may hybridize to each other.

Methods

Fluorophore Conjugation

Labeling procedure refers to the protocol given by Invitrogen, with minor modifications. More specifically, amine-modified DNA (16 μl, 100 μM) was mixed with phosphate buffer (10 μl, 0.4 M, pH 8.5), then one of the activated dye-esters (100 μg, ˜80 nmol) dissolved in DMSO was added. In this mixture, the final concentration for DNA was about 40 μM, and the molar ratio of ester and amine was 50:1. After incubation at 28° C. overnight, the mixture was treated by ethanol precipitation, followed by reverse-phase HPLC purification (5-40% MeCN in 0.1 M TEAA over 15 minutes) on an Agilent 1200 Series. Samples within the corresponding peak with absorption maximum at 260 nm were collected, freeze-dried, and re-dissolved in 200 μl H₂O. The ultimate concentration was determined by a NanoDrop 1000 spectrophotometer before use.

Construction of the Assay and its Function for STV Detection

In a typical assay, strand A, B, and S with exact stoichiometric ratio of 1:1:1 were mixed in 1×[TAE-Mg²⁺] buffer (40 mM Tris-HAc (pH 8), 1 mM EDTA, 12.5 mM Mg(Ac)₂), as well as STV as target protein. The typical final concentration was 20 nM for each DNA component, and 250 nM for STV. An excess of STV was used to ensure monovalent binding, but is not necessary for practical sensing. For convenience, the mixture was incubated at room temperature (RT) overnight before measurement, although kinetics data has shown that 3 hours is enough for the system to reach near equilibrium (data not shown).

FRET Measurement by Spectrofluorometer.

70 μl of the assay mixture was pipetted into a quartz cuvette, which was washed by 1×[TAE-Mg²⁺] twice between different samples. Fluorescence measurements were done with a scanning spectrofluorometer (Fluoro-Max-3, HORIBA Jobin Yvon Inc.). Excitation was performed at 530 nm for Alexa555. FRET efficiencies were calculated as E=I_{A}/(I_{A}+I_{D}), where I_{A} is the acceptor peak fluorescence intensity (667 nm for Alexa647) and I_{D} is the donor peak fluorescence intensity (566 nm for Alexa555).

Gel Experiment and Typhoon Scanning.

15 μl of each sample which was used for bulk FRET measurement was mixed with 3 μl of 6× Gel Loading Dye (NEB) before being loaded into the wells of a 6% non-denaturing polyacrylamide gel electrophoresis (PAGE) gel (acrylamide/bisacrylamide; 19:1) and eluted in a fridge (70 V, 1.5 h). 1× TBE-Mg²⁺ ([Mg²⁺]=12.5 mM) was used for making both the gel itself and the running buffer.

After electrophoresis, the gel was scanned using a Typhoon 9400 (Amersham Biosciences) in fluorescence mode, without staining. Red laser (633 nm) and 670 nm band-pass filter (transmits light between 655 nm and 685 nm and has a transmission peak centered at 670 nm) were chosen as the combination for excitation and emission of Alexa647 dye respectively. A typical scanning time for a 8.3×7.3 cm mini-gel was 3 min, when using 200 μm of pixel size. Afterwards, the gel was analyzed by ImageQuant TL 7.0 (GE Healthcare). Lane creation and band detection were done manually.

Results

Without STV, the hybridization events between the three DNA strands will reach an equilibrium, in which there should be more BS duplex than AS duplex, since B has a 4 nt toehold to bind with S while A only has a 3 nt toehold. With STV, which can bind with B through biotin-STV interaction, the original equilibrium will be shifted since the bulkiness of STV will hinder the sensitive branch migration process only through which B can displace A to hybridize with S, thus there should be less BS duplex and more AS duplex in the sample.

Two fluorophores forming a FRET (Förster resonance energy transfer) pair were put on A and S respectively, to give off FRET signal representing the population of AS duplex in solution (see also FIG. 3). Nearly a doubling of the normalized FRET efficiency was revealed after adding STV (FIG. 4a), and their widely divergent typical raw fluorescence data are also shown (FIG. 4b). In addition, a titration curve was made and is shown in FIG. 4c, showing the possibility of quantification detection and a sensitivity of sub-nanomole. Further evidence comes from the non-denaturing PAGE gel (FIG. 4d), where each component in the solution can be observed separately. It's noteworthy that the selected excitation and emission wavelength ensures that the band intensity here only reflects the amount of A. The result completely conforms to the expectation that much fewer single-stranded A (ssA) are left and meanwhile more AS duplex is formed in the presence of STV (FIG. 4d, one-biotin).

In this example, two biotins were also put on one B strand. This makes use of the fact that STV has tetravalent binding ability, so the biotinylated B will curl up and wrap around the STV by a two-site interaction (FIG. 4d inset). In this way, the toehold cannot function at all, therefore even fewer BS and more AS are expected. Indeed, the change is more impressive than the one-biotin system after addition of STV (FIG. 4c, two-biotin).

Comments

In addition to the results above, the following observations have been made (data not shown):
1. A fluorophore-quencher pair has been tried as an alternative reporter system instead of FRET. This also provided suitable results.
2. Two negative controls have been tested: STV added in a system without biotin, other proteins (thrombin or other antibodies) added in a biotinylated system. Neither exhibits significant FRET change.
3. The sensitivity of this sensing assay relies on the sensitivity of the instruments used for measuring the FRET value. In studies performed thus far, as low as 1 nM STV as the target protein has been successfully detected by using an assay with 1 nM DNA as the final concentration.

Conclusion

We have successfully used the biotin-STV interaction as a model system to show that the assay which is based on the equilibrium of three DNA strands has the great potential of detecting specific target proteins, as well as small molecules. Both bulk FRET data and Typhoon-scanning gel experiment validates our design, and the signal can be doubled (or halved depending on the design) after adding STV.

### Example 2

Detection of a Small Molecule: Biotin

By applying the inhibition strategy (FIG. 5; Strategy 2) in the system described in example 1, biotin can be detected. In this experiment, biotin as the target being detected was first mixed with a predetermined amount of STV, then the whole mixture was added into the standard assay. In the presence of free biotin, all the active binding sites on STV will be occupied, thus it can't have an effect on the DNA equilibrium. This signal-off detection method was tested by using 14 nM of DNA, 20 nM of STV, and a series of concentrations of biotin. A titration curve is shown in FIG. 4e. The molar ratio between biotin and STV is consistent with the tetravalent property of STV.

Methods

An assay setup identical to the assay in example 1 before streptavidin addition was used, using 14 nM DNA. Biotin in varying concentrations was premixed with the streptavidin (20 nM) first. After incubation at RT for 3 hours, the mixture was added into the normal assay and incubated at RT overnight in the dark.

Results

In the absence of biotin as target molecule, STV shows the same effect on the equilibrium as before. If the sample contains biotin, these free biotins will block the binding sites on STV, making the STV incapable of functioning on the assay. This signal-off detection method provided a smooth calibration curve for the detection of biotin and the results reflect the tetravalence of STV (FIG. 4e).

### Example 3

Specificity of the Biotin Detection Assay

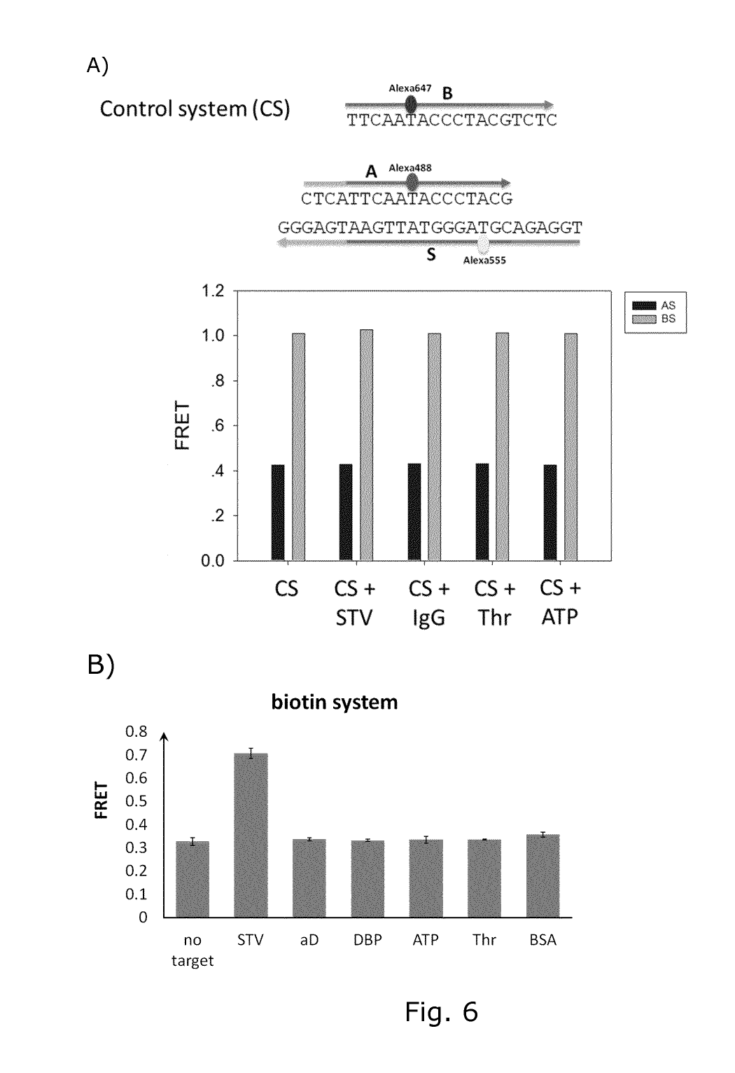

A control system may be comprised of three DNA strands with two FRET pairs, but without any labelled binding moiety. Adding any of the targets streptavidin, IgG, thrombin or ATP into this system will not result in detectable signal change (FIG. 6a), which not only validates the design, that it is the binding event which shifts the equilibrium, but also serves as a proof of good specificity of this system.

The specificity was also tested by adding a variety of targets in the biotin system described in example 1, and none of them showed unspecific detectable effect (FIG. 6b). This assay was also validated by adding STV into a control system without ligand and no FRET signal change was observed.

### Example 4

Anti-Digoxigenin (aD) Detection in Buffer

Materials

Three DNA strands are used in this experiment, named A, B, and S respectively. A and S are with internal amine modification, which is used as a handle for Alexa fluorophore labeling, while B has an internal digoxigenin modification. The sequences are given below (underlining indicates the toehold regions):

| name | sequence | modification |
|---|---|---|
| A- | CTCATTCAA1ACCCTACG | 1: Int Amino Modifier C6 |
| SEQ ID NO: 7 | | dT |
| B- | TTCAATACCC2ACGTCTC | 2: 5-Aminoallyl-dU |
| SEQ ID NO: 8 | | |
| S- | TGGAGACG1AGGGTATTGAATGAGGG | 1: Int Amino Modifier C6 |
| SEQ ID NO: 6 | | dT |

All the oligonucleotides for A, B and S were synthesized in-house on a MerMade-12 oligonucleotide synthesizer from Bioautomation. Following the synthesis the DNA strands were TOP-cartridge purified and ethanol precipitated.

Alexa Fluor® 647 Succinimidyl Ester and Alexa Fluor® 555 Succinimidyl Ester was purchased from Invitrogen.

The 5-Aminoallyl-dU phosphoramidite was purchased from Berry & Associates.

Anti-digoxigenin was purchased from Roche.

All the other reagents were purchased from Sigma-Aldrich.

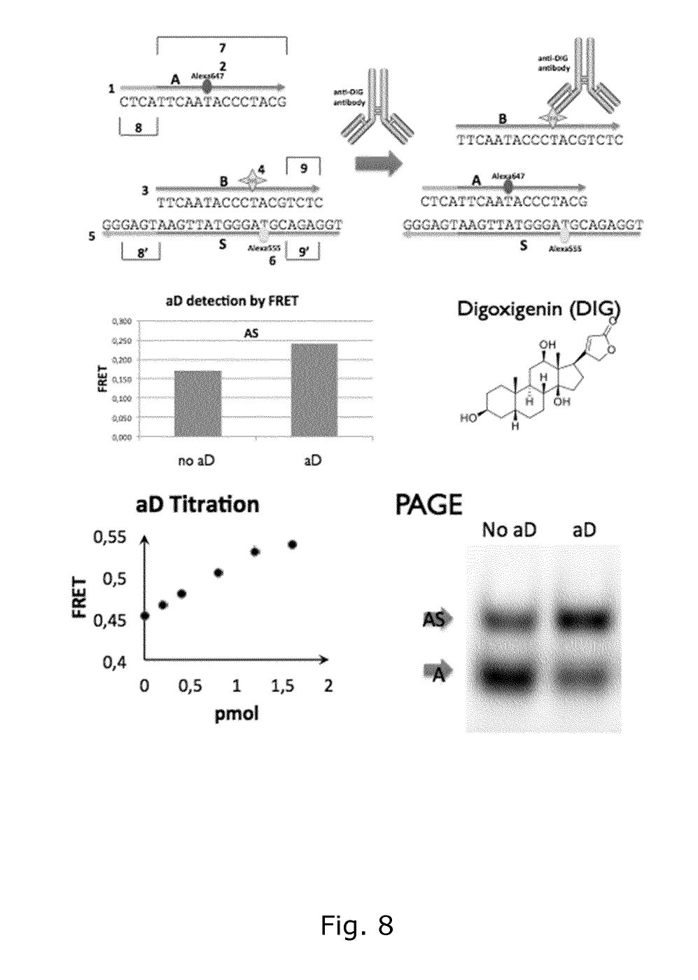

FIG. 8A illustrates how the oligonucleotides may hybridize to each other.

Methods

Fluorophore Conjugation

Same as example 1.

Digoxigenin Conjugation

Amine-modified DNA (100 μL, 50 μM) was added to the activated digoxigenin ester (DIG-NHS) in DMF (100 μL, 150 nmol), then triethylamine (2 μL) was added to this. In this mixture the final concentration of the DNA is 25 μM, and the molar ratio of ester and amine is 30:1. After incubation at 25° C. for 1 hour, the mixture was treated by ethanol precipitation, followed by reverse-phase HPLC purification (5-40% MeCN in 0.1 M TEAA over 15 minutes) on an Agilent 1200 Series. Samples with the corresponding peak with absorption maximum at 260 nm were collected, freeze-dried, and re-dissolved in 200 μL H₂O. The ultimate concentration was determined by a NanoDrop 1000 spectrophotometer before use.

Construction of the Assay and Its Function for aD Detection

Same as example 1, with aD instead of STV as target protein, and with only a 2 times excess of anti-digoxigenin (20 nM of each DNA component, and 40 nM of aD).

FRET Measurement by Spectrofluorometer

The sample preparation and FRET measurements are the same as example 1

Gel Experiment and Typhoon Scanning

Same as example 1.

Results

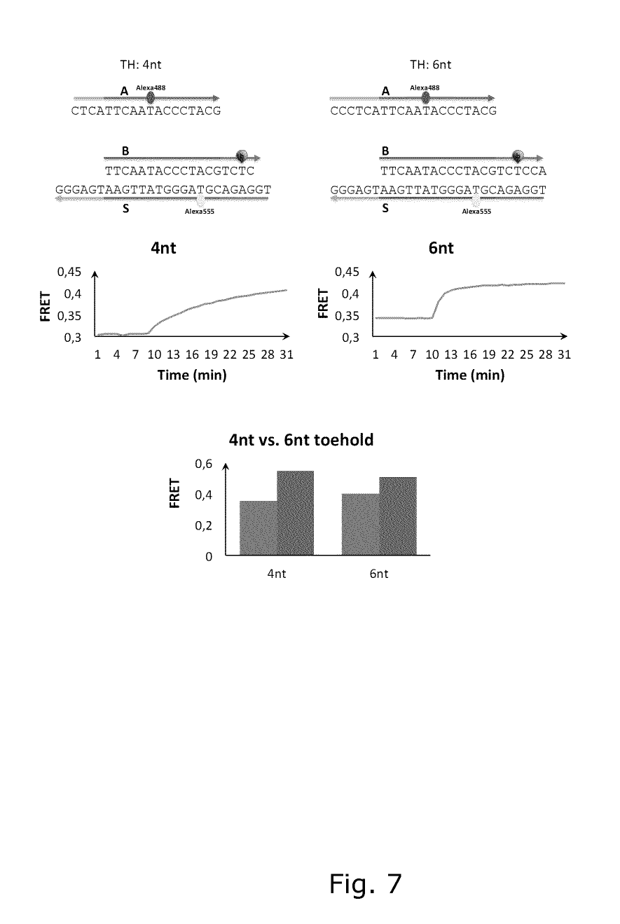

The results obtained are shown in FIG. 7. A FRET value reflecting a 30% population increase of AS in the presence of aD is observed. Titration with various concentrations of aD is shown in FIG. 8D. The complementary detection method of aD by electrophoresis is shown in FIG. 8E

Example 5

Detection of Anti-Digoxigenin (aD) in Human Plasma.

Materials

Three DNA strands are used in this experiment, named A, B, and S. A and S are with internal amine modification, which is used as a handle for Alexa fluorophore labeling, while B has an internal digoxigenin (DIG) modification. The sequences are given below (underlining indicates the toehold regions):

| name | sequence | modification |
|---|---|---|
| A- | CTCATTCAA1ACCCTACG | 1: Int Amino Modifier C6 |
| SEQ ID NO: 7 | | dT |
| B- | TTCAATACCC2ACGTCTC | 2: 5-Aminoallyl-dU |
| SEQ ID NO: 8 | | |
| S- | TGGAGACG1AGGGTATTGAATGAGGG | 1: Int Amino Modifier C6 |
| SEQ ID NO: 6 | | dT |

All the oligonucleotides for A, B and S were synthesized in-house on a MerMade-12 oligonucleotide synthesizer from Bioautomation. Following the synthesis the DNA strands were TOP-cartridge purified and ethanol precipitated.

Alexa Fluor® 647 Succinimidyl Ester and Alexa Fluor® 555 Succinimidyl Ester were purchased from Invitrogen.

The 5-Aminoallyl-dU phosphoramidite was purchased from Berry & Associates.

Anti-digoxigenin was purchased from Roche.

Human whole blood was donated by the blood bank at Aarhus University Hospital in Skejby, Denmark.

All the other reagents were purchased from Sigma-Aldrich.

Methods

Fluorophore Conjugation Same as example 1.

Digoxigenin Conjugation

Same as example 4.

Human Plasma Preparation

The human whole blood was EDTA buffered by the hospital upon collection from the donor, and the plasma was separated out of the whole blood sample within 30 minutes of the blood collection. This was done by centrifugation at 3000 g for 15 minutes at 20° C. The top layer, which constitutes the plasma, was carefully pipetted off and immediately frozen in smaller aliquots.

Construction of the Assay and Its Function for aD Detection

Same as example 5. However, the samples were added 15% v/v pure plasma premixed with antibody of interest (aD) prior to overnight incubation.

FRET Measurement by Spectrofluorometer

The sample preparation and FRET measurements are the same as example 4. However, the spectra containing human plasma was reference subtracted using a sample containing only human plasma in TAE-Mg buffer (10 μL plasma in 60 μL 1× [TAE-Mg²⁺]) as a reference.

Results

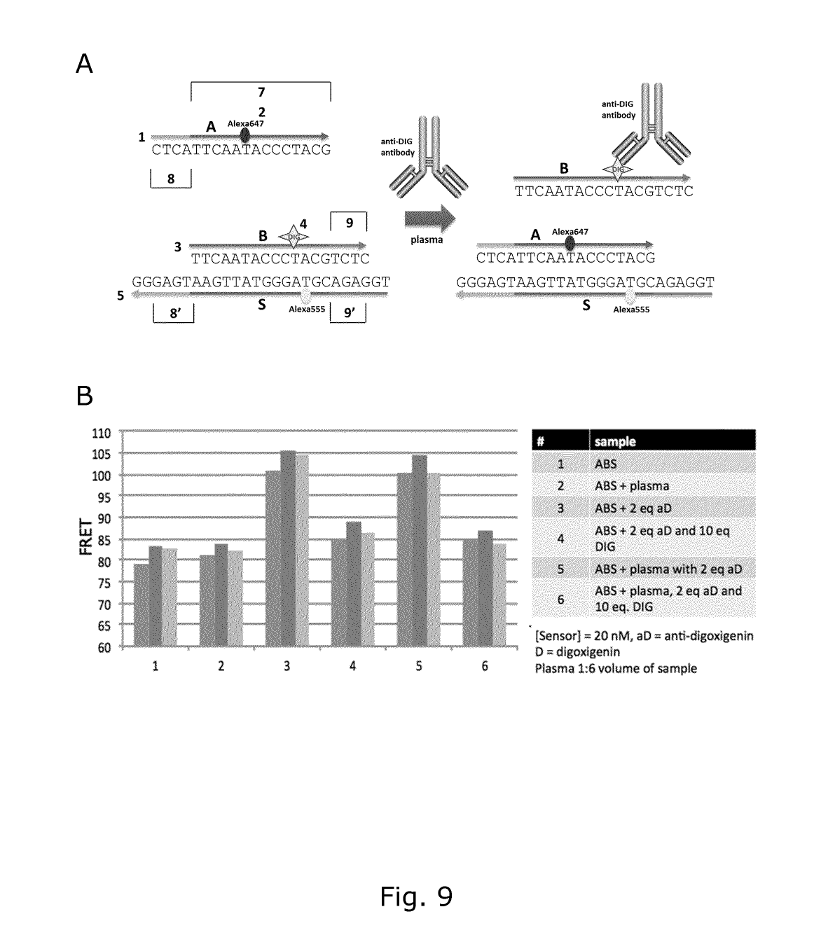

The results obtained from three independent experiments are shown in FIG. 9. As can be observed the AS percentage before the addition of aD in buffer and in the plasma are identical (samples 1 and 2 in B) Upon addition of 2 eq. aD the Fret increase is only incrementally lower in plasma (sample 5) compared to in buffer (sample 3). Results for detection of digoxigenin (DIG) by strategy 3 in buffer (sample 4) and plasma (sample 6) are also included; this will be described in more detail in example 8

### Example 6

Detection of Anti-Digoxigenin (aD) in Human Saliva.

Materials

Three DNA strands are used in this experiment, named A, B, and S. A and S are with internal amine modification, which is used as a handle for Alexa fluorophore labeling, while B has an internal digoxigenin (DIG) modification. The sequences are given below (underlining indicates the toehold regions):

| name | sequence | modification |
|---|---|---|
| A- | CTCATTCAA1ACCCTACG | 1: Int Amino Modifier C6 |
| SEQ ID NO: 7 | | dT |
| B- | TTCAATACCC2ACGTCTC | 2: 5-Aminoallyl-dU |
| SEQ ID NO: 8 | | |
| S- | TGGAGACG1AGGGTATTGAATGAGGG | 1: Int Amino Modifier C6 |
| SEQ ID NO: 6 | | dT |

All the oligonucleotides for A, B and S were synthesized in-house on a MerMade-12 oligonucleotide synthesizer from Bioautomation. Following the synthesis the DNA strands were TOP-cartridge purified and ethanol precipitated.

Alexa Fluor® 647 Succinimidyl Ester and Alexa Fluor® 555 Succinimidyl Ester were purchased from Invitrogen.

The 5-Aminoallyl-dU phosphoramidite was purchased from Berry & Associates.

Anti-digoxigenin was purchased from Roche.

Human saliva was collected from a human donor that had fasted for one hour.

All the other reagents were purchased from Sigma-Aldrich.

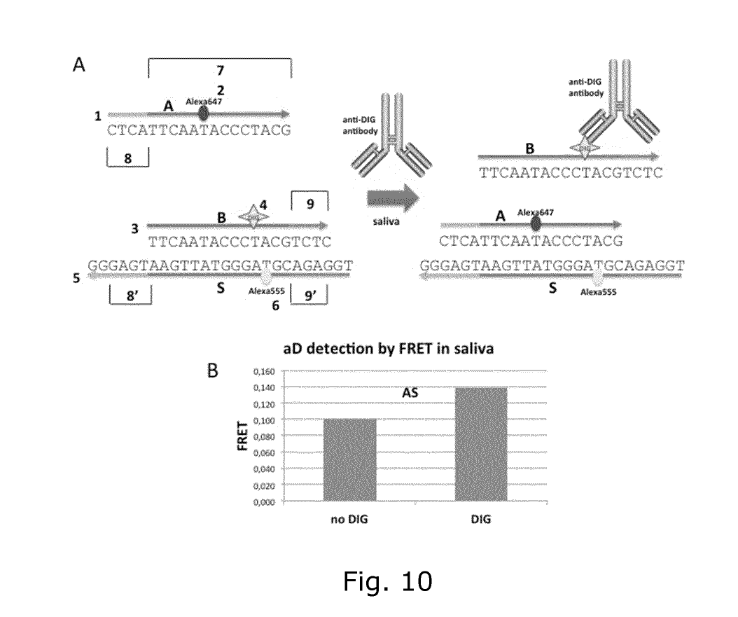

FIG. 10 illustrates how the oligonucleotides may hybridize to each other.

Methods

Fluorophore Conjugation

Same as example 1.

Digoxigenin Conjugation

Same as example 4.

Human Saliva Preparation

Saliva was collected into a Falcon tube over 1 hour from a male human who had been fasting for 1 hour. The saliva was vortexed thoroughly for 1 min, followed by centrifugation at 4° C., at 10,000 g for 10 min. The liquids were separated from the solids, and the saliva was filtered through a 100k Amicon Ultra-0.5 mL Centrifugal Filter.

Construction of the Assay and Its Function for aD Detection

Same as example 5. However, the samples were added 15% v/v filtered saliva premixed with antibody of interest (aD) prior to overnight incubation.

FRET Measurement by Spectrofluorometer

The sample preparation and FRET measurements are the same as example 7. However, the spectra containing human plasma was reference subtracted using a sample containing only filtered saliva in TAE-Mg buffer (10 μL plasma in 60 μL 1× [TAE-Mg²⁺]) as a reference.

Results

The results are comparable to example 7, with a FRET value reflecting a 28% population increase of AS in the presence of aD in human saliva, also shown in FIG. 10

### Example 7

Detection of aD with Multiple DIG Modifications on DNA

Materials

Three DNA strands are used in this experiment, named A, B, and S. A and S are with internal amine modification, which is used as a handle for Alexa fluorophore labeling, while B has four internal digoxigenin modifications (but not limited to four internal digoxigenin modifications). The sequences are given below (underlining indicates the toehold regions):

| name | sequence | modification |
|---|---|---|
| A- | CTCATTCAA1ACCCTACG | 1: Int Amino Modifier C6 |
| SEQ ID NO: 7 | | dT |
| B- | T2CAA2ACCC2ACG2CTCC | 2: 5-Aminoallyl-dU |
| SEQ ID NO: 9 | | |
| S- | TGGAGACG1AGGGTATTGAATGAGGG | 1: Int Amino Modifier C6 |
| SEQ ID NO: 6 | | dT |

All the oligonucleotides for A, B and S were synthesized in-house on a MerMade-12 oligonucleotide synthesizer from Bioautomation. Following the synthesis the DNA strands were TOP-cartridge purified and ethanol precipitated.

Alexa Fluor® 647 Succinimidyl Ester and Alexa Fluor® 555 Succinimidyl Ester were purchased from Invitrogen.

The 5-Aminoallyl-dU phosphoramidite was purchased from Berry & Associates.

Anti-digoxigenin was purchased from Roche.

All the other reagents were purchased from Sigma-Aldrich.

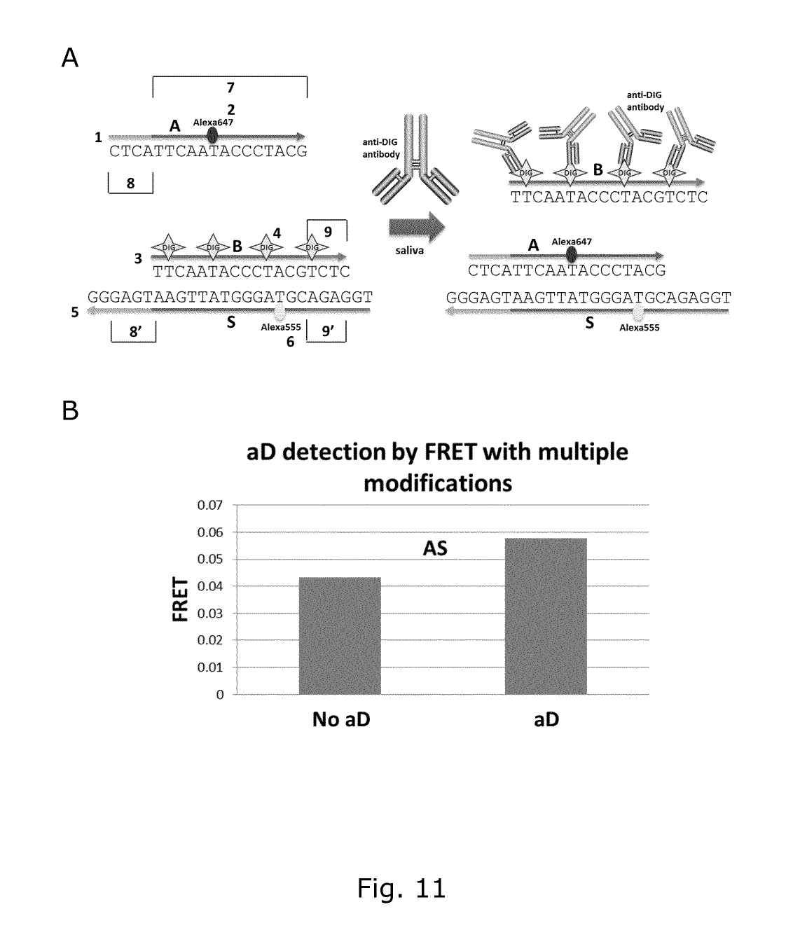

FIG. 11 illustrates how the oligonucleotides may hybridize to each other.

Methods

Fluorophore Conjugation

Same as example 1.

Digoxigenin Conjugation

Same as example 4.

Construction of the Assay and Its Function for aD Detection

Same as example 1, with aD instead of STV as target protein, and with an 8 times excess of anti-digoxigenin (20 nM of each DNA component, and 160 nM of aD).

FRET Measurement by Spectrofluorometer

The sample preparation and FRET measurements are the same as example 1

Gel Experiment and Typhoon Scanning

Same as example 1.

Results

The results are comparable to example 4, with a FRET value reflecting a 33% population increase of AS in the presence of aD.

### Example 8

Detection of Digoxigenin (DIG) by Strategy 2 and 3.

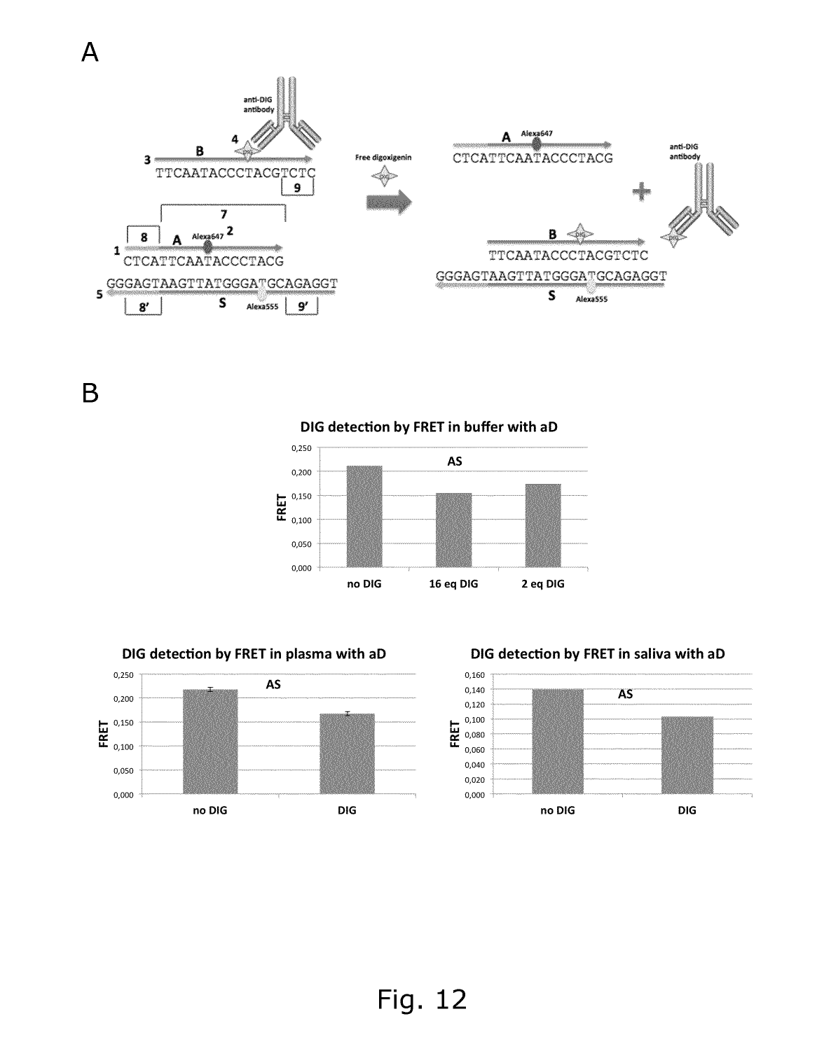

FIG. 12 illustrates how the oligonucleotides may hybridize to each other.

Materials

As examples 4, 5 and 6, with the addition of digoxigenin from Sigma-Aldrich.

Methods

Fluorophore Conjugation

Same as example 1.

Digoxigenin Conjugation

Same as example 4.

Construction of the Assay and Its Function for DIG Detection

Same as example 4, 5 and 6, with free DIG being additionally added to the samples in varying concentrations for a competitive assay.

FRET Measurement by Spectrofluorometer

The sample preparation and FRET measurements are the same as example 7, 9 and 10, with the respective signal adjustments being performed with and without the addition of human plasma and saliva.

Results

The results are comparable to example 4, 5 and 6, with a FRET value reflecting only a 0.5% population increase of AS in the presence of 320 nM DIG and an increase of 11% AS population in the presence of 40 nM DIG, compared to the normal 30% increase without the presence of DIG. In the case of human plasma and saliva containing 40 nM DIG, the AS population increases are 5% and 2% respectively, compared to the 26% and 28% increase without DIG. Results are also shown in FIG. 12.

### Example 9

Detection of the Vitamin D-Binding Protein (DBP) and Vitamin D (VD).

Materials

Three DNA strands are used in this experiment, named A, B, and S. A and S are with internal amine modification, which is used as a handle for Alexa fluorophore labeling, while B has an internal vitamin D modification. The sequences are given below (underlining indicates the toehold regions):

| name | sequence | modification |
|---|---|---|
| A- | CTCATTCAA1ACCCTACG | 1: Int Amino Modifier C6 |
| SEQ ID NO: 7 | | dT |
| B- | TTCAATACCC2ACGTCTC | 2: 5-Aminoallyl-dU |
| SEQ ID NO: 16 | | |
| S- | TGGAGACG1AGGGTATTGAATGAGGG | 1: Int Amino Modifier C6 |
| SEQ ID NO: 6 | | dT |

All the oligonucleotides for A, B and S were synthesized in-house on a MerMade-12 oligonucleotide synthesizer from Bioautomation. Following the synthesis the DNA strands were TOP-cartridge purified and ethanol precipitated.

Alexa Fluor® 647 Succinimidyl Ester and Alexa Fluor® 555 Succinimidyl Ester were purchased from Invitrogen.

The 5-Aminoallyl-dU phosphoramidite was purchased from Berry & Associates.

The activated vitamin D ester was synthesized in-house in a two-step manner from cholecalciferol.

All the other reagents were purchased from Sigma-Aldrich.

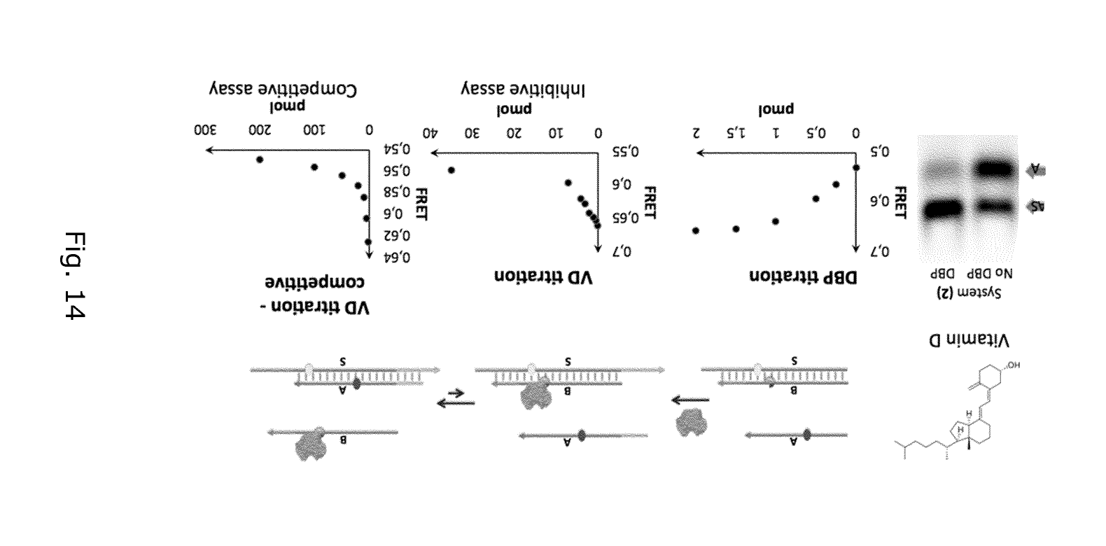

FIG. 14 illustrates how the oligonucleotides may hybridize to each other.

Methods

Fluorophore Conjugation

Same as example 1.

Vitamin D Conjugation

Same as DIG-NHS in example 4, with the activated vitamin D ester replacing DIG-NHS.

Construction of the assay and its function for DBP detection

Same as example 4, with DBP instead of aD as target protein.

FRET Measurement by Spectrofluorometer

The sample preparation and FRET measurements are the same as example 1.

Gel Experiment and Typhoon Scanning

Same as example 1.

Results

The results are comparable to example 1, but with a FRET value reflecting a 20% population increase of AS in the presence of DBP, also shown in FIG. 14.

Furthermore, both the inhibitive and competitive strategies were used for detection of Vitamin D. Various concentrations of Vitamin D were added into the assay mixture before (Inibitory strategy 2) or after (Competitive strategy 3) addition of DBP protein. Both methods were successful for detection of Vitamin D, as it appears from the calibration curves in FIG. 14.

### Example 10

ATP Detection as Examples of Aptamer Systems.

Materials

Three DNA strands are used in this example, named A, B, and S respectively. A and S are with internal amine modification, which is used as a handle for fluorophore labeling. B doesn't have any additional modification, but includes a sequence of ATP aptamer at the 3′ end. The sequences are given below (italic indicates toehold regions; underlining indicates aptamer regions):

| name | sequence | modification |
|---|---|---|
| A- | CTCATTCAA1ACCCTACG | 1: Int Amino Modifier C6 |
| SEQ ID NO: 10 | | dT |
| B- | TTCAATACCCTACGACCTGGGGGAGT | |
| SEQ ID NO: 11 | ATTGCGGAGGAAGGT | |
| S- | CCCCAGGTCG1AGGGTATTGAATGAG | 1: Int Amino Modifier C6 |
| SEQ ID NO: 12 | GG | dT |

All the oligonucleotides were purchased from DNA Technology A/S, Denmark. RP-HPLC purification was done by the company directly after synthesis.

Alexa Fluor® 647 Succinimidyl Ester and Alexa Fluor® 555 Succinimidyl Ester were purchased from Invitrogen.

All the other reagents were purchased from Sigma-Aldrich.

Methods

Fluorophore Conjugation.

Same as Example 1.

Construction of the Assay and Its Function for aF Detection.

Same as Example 1, with a range of concentrations of ATP molecules instead of STV.

FRET Measurement by Spectrofluorometer.

Same as Example 1.

Results

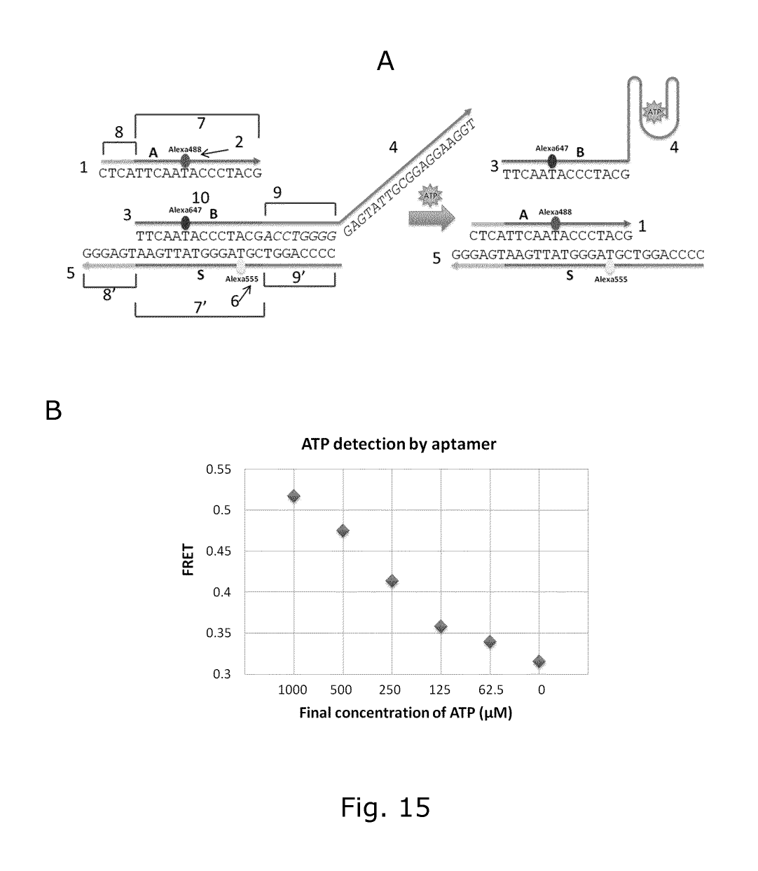

The design of this example is inspired by the so-called structure-switching aptamer, which typically undergoes target-induced switching between a DNA duplex and an aptamer-target complex. Here, part of the aptamer (8 nt) serves as the toehold on B, which can hybridize with the toehold on S, resulting a duplex dominant in solution since A has shorter toehold (4 nt). When the target (ATP) is present, the aptamer-target binding is strong enough to outcompete the hydrogen bond in duplex, leaving no functional toehold on B. Hence, AS duplex will account for the majority. The scheme is shown in FIG. 15a.

A titration curve was made in micromole scale (FIG. 15b). Up to 60% increase in FRET for AS duplex can be observed in the presence of 1 mM ATP. The concentration of each DNA component used here was 20 nM.

Conclusion

In this example, we combined the toehold exchange reaction system with structure-switching aptamer design to achieve target detection through aptamer-target binding. Without amplification, the FRET signal change has already been significant enough to observe, and even quantification is possible. It is noted that the aptamer system is functional with the aptamer region being positioned either at the 5′ end or at the 3′ end.

### Example 11

Streptavidin (STV) Detection Using the DNA Peroxidase Signalling System

Materials

Three DNA strands are used in this example, named A, B, and S respectively, among which only B has a modification. The sequences are given below (italic indicates the toehold region; underlining indicates the G-rich region for DNA peroxidase):

| name | sequence | modification |
|---|---|---|
| A- | TGGGTATTCAATACCCTACG | |
| SEQ ID NO: 13 | | |
| B-A- | TTCAATACCC2ACGTC2C | 2: Int Biotin dT |
| SEQ ID NO: 14 | | |
| S-A- | TGGAGACGTAGGGTATTGAATTGGGCGGGTGGGT | |
| SEQ ID NO: 15 | | |

All the oligonucleotides were purchased from DNA Technology A/S in Denmark. RP-HPLC purification was done by the company directly after synthesis.

All the reagents were purchased from Sigma-Aldrich.

Methods

Construction of the Assay and Its Function for STV Detection

In a typical assay, strand A, B, and S with the exact stoichiometric ratio of 1:1:1 were mixed in 1×[TAE-Mg²⁺] buffer (40 mM Tris-HAc (pH 7), 1 mM EDTA, 12.5 mM Mg(Ac)₂), as well as STV as target protein. The mixture was incubated at room temperature (RT) for 3 hours, before adding hemin (Ferriprotoporphyrin IX chloride), ABTS (2,2′-azino-bis(3-ethylbenzothiazoline-6-sulphonic acid)) and H₂O₂ (hydrogen peroxide) into it. The typical final concentration was 200 nM for the biotinylated DNA, 400 nM for STV, 2 μM for hemin, and 2 mM for ABTS and H₂O₂.

Absorbance Measurement by Nanodrop

After incubation at room temperature for half an hour, 1.5 μl of each assay mixture was pipetted onto the pedestal of a Nanodrop 1000 Spectrophotometer (Thermo Fisher Scientific Inc.), and was measured in UV-Vis mode. The absorbance at 420 nm was recorded.

Results

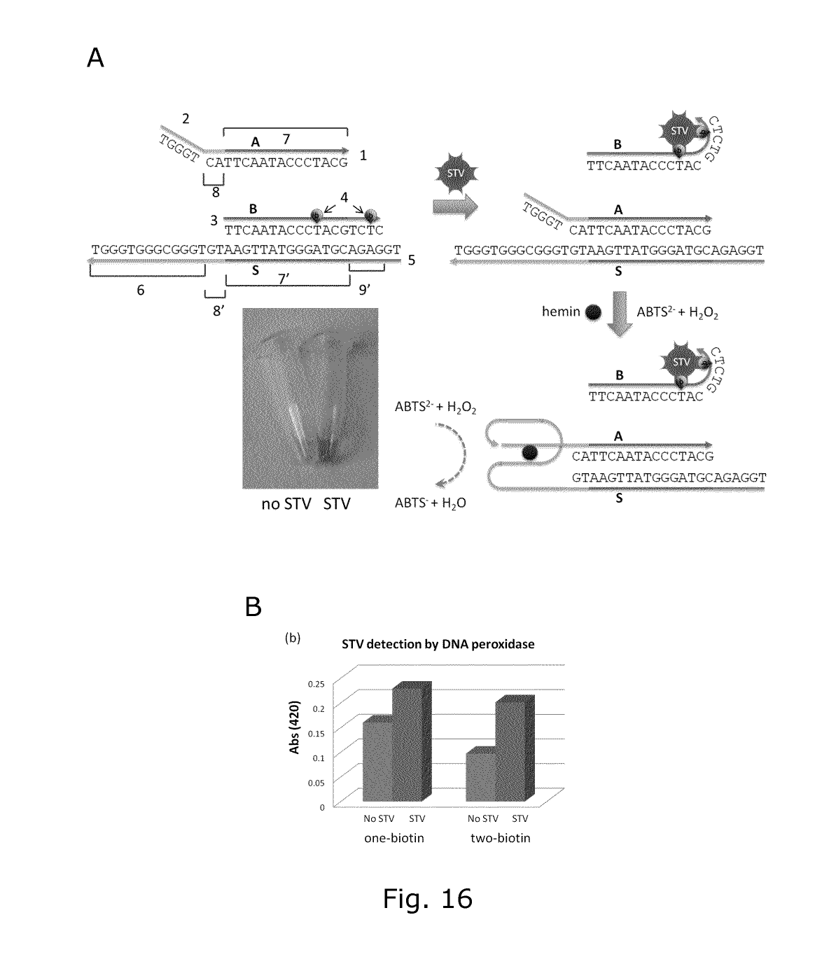

In this example, we give an example of incorporating a split DNAzyme at the end of both A and S, which can recover its catalytic ability when they are brought together by hybridization between A and S. The scheme of a two-biotin system is shown in FIG. 16.

Without STV, S tends to bind with B, since B has a longer toehold. Low peroxidase activity can be found due to separate parts of the G-quadruplex. With STV, strand B loses its toehold, which is embedded in the protein surface, thus the formation of AS duplex results in a fully functional G-quadruplex. Green color would appear after adding necessary reactants such as H₂O₂, ABTS²⁻ and hemin, and the max absorbance at 420 nm can be recorded.

Significant absorbance change can be observed in FIG. 16, and the difference is even distinguishable with the naked eye. Notice that the change in the one-biotin system is also pronounced, although it's hard to discern by direct visualization due to stronger background.

Conclusion

We have employed a new reporter system to provide an optical signal for our assay. The advantage of this system is that it only needs pure DNA extension instead of covalent labeling, and its catalytic and colorimetric feature makes the results detectable even directly by eye. This method may also be employed in a one strand system as described in example 3.

### Example 12

One-Strand System Combined with DNA G-Quadruplex Peroxidase for Streptavidin (STV) Detection.

Materials

Only one DNA strand is used in this experiment, named L. An internal amine group is modified at a specific position, which is used as a handle for further labeling. Its sequence is given below (italic indicates toehold regions; underlining indicates G-rich regions for DNA peroxidase):

| name | sequence | modification |
|---|---|---|
| L- | TGGGTCAATACCCTACGATACCC1ACG | 1: Int Amino Modifier C6 |
| SEQ ID NO: 17 | TCTCTTTTTTGAGACGTAGGGTATTGT | dT |
| | GGGCGGGTGGGT | |

This oligonucleotide was purchased from DNA Technology A/S, Denmark. RP-HPLC purification was done by the company directly after synthesis.

All the other reagents, including streptavidin, were purchased from Sigma-Aldrich.

FIG. 17 illustrates how the oligonucleotide may self-hybridize depending on the presence of an analyte.

Positions (nucleotides) 1-5 (2) is one-quarter of G4-DNA (DNA peroxidase). Positions 6-7 (8) is the first toehold. Positions 8-17 (7 or A) is the first branch migration region. Positions 18-27 (7or B) is the second branch migration region, which has the same sequence as the first branch migration region but endows a small molecule modification. Positions 28-31 (9) is the second toehold. Positions 32-37 (11) is the loop region providing flexibility. Positions 38-41 (9′) is the third toehold region which is complementary to the second toehold region. Positions 42-51 (7′ or S) is the third branch migration region, which is complementary to the first or second branch migration region. Positions 52-53 (8′) is the fourth toehold, which is complementary to the first toehold. Positions 54-66 (6) is the other three-quarters of DNA peroxidase.

Methods

Biotin Conjugation

This procedure is the same as fluorophore conjugation procedure in example 1, with biotin-NHS ester instead of dye-NHS ester.

Construction of the Assay and Its Function for STV Detection

In a typical assay, strand L was added into 1×[TAE-Mg²⁺] buffer (40 mM Tris-HAc (pH 7), 1 mM EDTA, 12.5 mM Mg(Ac)₂), as well as STV as target protein. The mixture was incubated at room temperature (RT) for 3 hours, before adding hemin (Ferriprotoporphyrin IX chloride), ABTS (2,2′-azino-bis(3-ethylbenzothiazoline-6-sulphonic acid)) and H₂O₂ (hydrogen peroxide). A typical final concentration is 200 nM for the biotinylated DNA, 400 nM for STV, 2 μM for hemin, and 2 mM for ABTS and H₂O₂.

Absorbance Measurement by the Nanodrop Spectrophotometer

After one hour of incubation at room temperature, 1.5 μl of each assay mixture was pipetted onto the pedestal of a Nanodrop 1000 Spectrophotometer (Thermo Fisher Scientific Inc.), and was measured in UV-Vis mode. The absorbance at 420 nm was recorded.

Results

The idea of this design is to simply incorporate all three oligonucleotides into one long DNA strand, which not only circumvents the stoichiometry issue but also has the potential of speeding up the kinetics of strand displacement due to intramolecular reactions. To avoid the difficulty of making three modifications on one DNA strand, another reporter system called split DNA peroxidase was chosen to replace the FRET system used in previous designs. The exact sequence of the DNA peroxidase and its way of splitting are described in Shimron, S.; Wang, F.; Orbach, R.; Willner, I. Amplified detection of DNA through the enzyme-free autonomous assembly of hemin/G-quadruplex DNAzyme nanowires. Anal Chem 2012, 84, 1042-1048. Gratifyingly, this signaling system is also functional for a detection system according to the present invention which is based on hybridization equilibriums, even in the case where the oligonucleotides are covalently linked.

The long DNA strand in this example is comprised of several regions (FIG. 18). From 5′ to 3′ in sequence are: one quarter of the G-quadruplex, region a plus region b, which represents the original strand A, region b plus region d, which represents the original strand B thus having a biotin on it, loop e as a flexible hinge, region d* plus region b* plus region a*, which originates from strand S, and finally, three quarters of the G-quadruplex.

Without STV, d*b* will have the priority to hybridize with bd, since toehold d is longer than toehold a. In this state, the two G-quadruplex parts can't unite unless forcing the first region b into a bulge, which is not energetically favorable. With STV binding on the biotin on the second region b, the protein lump would hinder the local hybridization or branch migration, pushing the region b* to choose the first region b as its favored partner. Consequently, the two components of the G-quadruplex possess just the right position to form a complete G-tetrads, which has been found to have the ability to catalyze the H₂O₂-mediated oxidation of ABTS²⁻ to ABTS^{•-}, with the assistance of hemin. The kinetics of this reaction can be detected visually or by spectrophotometer since the product ABTS^{•-} has a green color. The conformational change process is illustrated in FIG. 18a.

The absorbance of the sample either with or without STV 1 h after adding ABTS as peroxidase substrate is given in FIG. 18b. An increase of more than 50% can be observed after adding STV. This proves the concept that STV binding results in reconfiguration and greater formation of G-tetrads. A control experiment has also been performed by mixing STV with a DNA strand with the same sequence but without biotin on it, in which case no signal change was found.

Conclusion

In this example, the detection system has been further simplified by using only one DNA strand labeled with a ligand to achieve target protein detection. Moreover, a new catalytic method is employed as a reporter as well as an amplification approach. The binding of STV in this example perturbs the intramolecular equilibrium of the strand displacement exchange reaction, which was reflected in the absorbance of the peroxidation product. We see this system as a potential alternative of ELISA (Enzyme-linked immunosorbent assay), which also targets proteins or small molecules (antibody and antigen especially) and uses color change as an indicator.

### Example 13

Effect of Toehold Length and the Position of the Binding Moiety.

To optimize the performance of the assay a range of tests have been performed (data not shown).

It is believed that the closeness of the melting temperature of A and B is important for the sensitivity of the equilibrium. Since A and B share the same branch migration region, the toehold region becomes the determinant in terms of magnitude of effect of a target-binding event in this system. We have tuned the toehold length on A for better discrimination between presence and absence of STV. The results showed that when A has 2 nt or 3 nt toehold, the effect of STV, i.e. the relative FRET change, is greatest. The reason that a 4 nt toehold for A is not optimal may be that the two modifications on B in this system change the equilibrium. We used a 3 nt toehold for A in the standard setup (Example 1), since A also has modification there.

Besides thermodynamics, we find that toehold length also has an effect on the kinetics of this system. A longer toehold can significantly increase the reaction rate, making it much faster to reach the equilibrium. This is consistent with the fact that a longer toehold will result in faster toehold hybridization, which is the rate-limiting step during the whole process.

The effect of the position of biotin was also investigated in this example. Biotin was labeled on one of the three typical positions on B: toehold region (TH), branch migration region (BM), and 3′ terminal (T3). It was found that biotin on the toehold was most sensitive to STV-binding in terms of FRET signal change, followed by biotin on BM. Biotin at the 3′ end of B only shows negligible STV effect. This is reasonable since DNA branch migration has been known to be very responsive to heterology in the presence of magnesium, so a small local environmental change might pose a substantial barrier. However, simple hybridization doesn't have this attribute.

1. An analyte detection system for detecting an analyte different from DNA and RNA, the system comprising at least a first oligonucleotide A, a second oligonucleotide B, and a third oligonucleotide S, wherein:
each of oligonucleotides A and B comprise a sequence that is complementary or partly complementary to a sequence on oligonucleotide S, and wherein oligonucleotides A and B compete for hybridization to oligonucleotide S in a dynamic equilibrium, and optionally wherein at least one of oligonucleotides A and B comprises a covalently linked binding moiety capable of interacting with an analyte different from DNA and RNA; and
at least one of oligonucleotides A and B, or a covalently linked binding moiety bound to said oligonucleotide, is capable of interacting with an analyte different from DNA and RNA, such that interaction of said analyte with the oligonucleotide or binding moiety results in a shift in the hybridization equilibrium, the shift in equilibrium providing a detectable signal.

2. The analyte detection system of claim 1, wherein the oligonucleotides are on separate nucleotide strands.

3. The analyte detection system of claim 1, wherein at least two of the oligonucleotides are partly or fully connected by covalent bonds.

4. The analyte detection system of claim 1, wherein oligonucleotide S comprises more than one hybridization domain, optionally wherein two or more hybridization domains are located on separate nucleotide strands.

5. The analyte detection system of claim 1, comprising a first oligonucleotide (1), a second oligonucleotide (3), and a third oligonucleotide (5); wherein:
the first or second oligonucleotide comprises a first group (2) forming a first part of a signaling system;
the third nucleotide comprises a second group (6) forming a second part of the signaling system;
at least one covalently linked binding moiety (4) is positioned on the first or second oligonucleotide;
wherein hybridization between the first or second oligonucleotide and the third oligonucleotide generates a signal or is able to catalyze generation of a signal different from when said first or second oligonucleotide and the third oligonucleotide are not hybridized;
and wherein the presence of the analyte changes the hybridization equilibrium of the detection system resulting in a change in signal.

6. The analyte detection system of claim 5, wherein:
the first oligonucleotide (1) comprises
a first toehold region (8) positioned at the 5′-side of a branch migration region (7);
the second oligonucleotide (3) comprises
a second toehold region (9) positioned at the 3′-side of a branch migration region (7); and
the third oligonucleotide (5) comprises
a first toehold region (8′);
a second toehold region (9′); and
a branch migration region (7′);
wherein:
the first toehold region (8) in the first oligonucleotide (1) and the first toehold region (8′) in the third oligonucleotide (5) comprise complementary sequences;
the branch migration region (7) in the first oligonucleotide (1) and the branch migration region (7′) in the third oligonucleotide (5) comprise a stretch of complementary nucleotides;
the second toehold region (9) in the second oligonucleotide (3) and the second toehold region (9′) in the third oligonucleotide (5) comprise a stretch of complementary nucleotides; and
the branch migration region (7) in the second oligonucleotide (3) and the branch migration region (7′) in the third oligonucleotide (5) comprise a stretch of complementary nucleotides.

7. The analyte detection system of claim 1, comprising:
a first oligonucleotide (1) comprising
a first toehold region (8) positioned at the 5′-side of a branch migration region (7);
optionally at least one covalently linked binding moiety (4);
optionally a first group (2), said first group forming a first part of a signaling system:
a second oligonucleotide (3) comprising
a second toehold region (9) positioned at the 3′-side of a branch migration region (7);
optionally at least one covalently linked binding moiety (4);
optionally a first group (2), said first group forming a first part of a signaling system;
a third oligonucleotide (5), comprising
a first toehold region (8′);
a second toehold region (9′);
a branch migration region (7′);
optionally at least one covalently linked binding moiety (4);
a second group (6), said second group forming a second part of the signaling system;
with the proviso that the first group (2) forming a first part of a signaling system is comprised in either the first oligonucleotide (1) and/or the second oligonucleotide (3);
with the proviso that at least one covalently linked binding moiety (4) is positioned on the first oligonucleotide (1) and/or the second oligonucleotide (3) and/or the third oligonucleotide (5);
wherein the first toehold region (8) in the first oligonucleotide (1) and the first toehold region (8′) in the third oligonucleotide (5) comprise complementary sequences;
wherein the branch migration region (7) in the first oligonucleotide (1) and the branch migration region (7′) in the third oligonucleotide (5) comprise a stretch of complementary nucleotides;
wherein the second toehold region (9) in the second oligonucleotide (3) and the second toehold region (9′) in the third oligonucleotide (5) comprise a stretch of complementary nucleotides;
wherein the branch migration region (7) in the second oligonucleotide (3) and the branch migration region (7′) in the third oligonucleotide (5) comprise a stretch of complementary nucleotides;
wherein hybridization between the first oligonucleotide (1) and the third oligonucleotide (5) generates a signal or is able to catalyze the generation of a signal different from when the first oligonucleotide (1) and the third oligonucleotide (5) are not hybridized, with the proviso that the first group (2) forming a first part of a signaling system is comprised on the first oligonucleotide (1); or
wherein hybridization between the second oligonucleotide (3) and the third oligonucleotide (5) generates a signal or is able to catalyze the generation of a signal different from the signal generated or catalyzed when the second oligonucleotide (3) and the third oligonucleotide (5) are not hybridized, with the proviso that the first group (2) forming a first part of a signaling system is comprised on the second oligonucleotide (3).

8. The analyte detection system of claim 1, where the signaling system formed by the first group (2) and the second group (6) is a quencher-fluorophore signaling system, a fluorophore-quencher signaling system, a FRET signaling system, a DNA peroxidase catalysis signaling system, or a quencher and singlet oxygen sensitizer; and optionally wherein the signaling system employs fluorescent nanoparticles.

9. The analyte detection system of claim 1, further comprising hemin and/or ABTS²⁻ and/or H₂O₂ and/or luminol.

10. The analyte detection system of claim 1, wherein the at least one covalently linked binding moiety (4) is selected from the group consisting of an organic molecule, an antibody, an antigen, an aptamer, biotin, and a hapten.

11. The analyte detection system of claim 1, wherein the covalently linked binding moiety (4) is an organic molecule having a molecular weight in the range 150-1500 Da, such as 150-1200 Da, such as 150-1000 Da, such as 150-800 Da, such as 150-600 Da, such as 150-400 Da, such as 150-300 Da, such as 300-1500 Da, such as 400-1500 Da, such as 600-1500 Da, such as 800-1500 Da, such as 1000-1500 Da, or such as 1200-1500 Da.

12. The analyte detection system of claim 1, wherein the binding moiety has its binding partner bound to the binding moiety.

13. The analyte detection system of claim 1, wherein the analyte is selected from the group consisting of proteins, peptides, organic molecules, antibodies, antigens and haptens.

14. The analyte detection system of claim 1, wherein the analyte is an organic molecule having a molecular weight in the range 150-1500 Da, such as 150-1200 Da, such as 150-1000 Da, such as 150-800 Da, such as 150-600 Da, such as 150-400 Da, such as 150-300 Da, such as 300-1500 Da, such as 400-1500 Da, such as 600-1500 Da, such as 800-1500 Da, such as 1000-1500 Da, or such as 1200-1500 Da.

15. The analyte detection system of claim 1, wherein the first oligonucleotide (1) is covalently linked to the second oligonucleotide (3) and the second oligonucleotide (3) is covalently linked to the third oligonucleotide.

16. A kit of parts comprising an analyte detection system according to claim 1.

17. A method for detection the presence or level of an analyte different from DNA and RNA in a sample, the method comprising
a) providing a sample comprising or suspected of comprising an analyte of interest;
b) providing the analyte detection system according to claim 1;
c) incubating the sample with the analyte detection system;
d) comparing the detected level of analyte to a reference level; and
e) determining the presence or level of analyte in the sample.

18. The method of claim 17, wherein the hybridization equilibrium between the first oligonucleotide and the third oligonucleotide and between the second oligonucleotide and the third oligonucleotide is shifted upon binding of an analyte to the binding moiety, and/or upon release of a binding partner to the binding moiety

19. The method of claim 17, wherein the sample is a biological sample. e.g. a blood sample such as a serum or plasma, a urine sample, a faeces sample, a biopsy sample, or a saliva sample.
